(19) United States
(12) Patent Application Publication
(10) Pub. No.: US 2007/0190651 A1
(43) Pub. Date: Aug. 16, 2007

(54) Myogenic Development and Protection of Stem Cells Against Inflammation and Apoptosis By Statins and Isoprenoid Pathway Inhibitors

(75) Inventor: Yong-Jian Geng, Houston, TX (US)

Correspondence Address: CONLEY ROSE, P.C.;David A. Rose, P. O. BOX 3267, HOUSTON, TX, 77253-3267, US

(73) Assignee: Board of Regents of the University of Texas System, Austin, TX (US)

(21) Appl. No.: 11/673,888

(22) Filed: Feb. 12, 2007

Related U.S. Application Data
(60) Provisional application No. 60/772,185, filed on Feb. 10, 2006.

(51) Int. Cl. C12N 5/08; C12N 5/06

(52) U.S. Cl. 435/325; 435/368

(57) ABSTRACT

A method of enhancing survival and differentiation of stem cells, and cells of myogenic lineage derived from said stem cells, when the cells are exposed to an inflammatory or apoptotic stimulus comprises culturing stem cells in vitro in a medium containing a statin, to produce statin-pretreated cells with enhanced resistance to inflammatory or apoptotic stimuli. Additionally, the statin-pretreated cells may be caused or allowed to differentiate into statin-pretreated cells of myogenic lineage (e.g., cells having a vascular or cardiac myocyte phenotype). The resulting inflammation- and apoptosis resistant cells may then be used for cell therapy, as in treating an ischemic or infarcted vessel or heart.

### CROSS-REFERENCE TO RELATED APPLICATION

This application claims the benefit under 35 U.S.C. § 119(e) of U.S. Provisional Patent Application No. 60/772185 filed Feb. 10, 2006, the disclosure of which is hereby incorporated herein by reference.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant Nos. R01HL59249 and R01HL69509 awarded by the National Institutes of Health.

### BACKGROUND OF THE INVENTION

1. Technical Field

The present invention generally relates to mammalian tissue repair and regeneration, and to cellular therapy and tissue engineering, for treatment of degenerative diseases such as atherosclerosis and heart failure. More particularly, the invention relates to the use of statins and isoprenoid pathway inhibitors in cellular therapy and tissue engineering, especially for protecting stem cells and myocytes against inflammatory injury and apoptosis, prolonging stem cell survival, and promoting their differentiation into myogenic cell lineages.

2. Description of Related Art

Inflammatory Response in Injured Tissue

Transplantation or mobilization of stem cells with growth factors has been emerging as a potentially novel therapy for regenerative medicine. However, one of the major challenges to successful stem cell therapy is the difficulty of stem cell survival and differentiation in the harsh microenvironment of diseased tissues or organs, such as infarcted or ischemic hearts with inflammation (1). Tissues in response to inflammatory stimulation can release radicals such as nitric oxide (NO), a gaseous signaling molecule that plays an important role in regulation of myocardial metabolism and function (2). Through NO synthases (NOS), cardiovascular cells can transiently synthesize large quantities of NO which, in turn, regulate the heart contractility (3). Exposure to NO at high levels may cause dysfunction of mitochondria (4,5), and trigger apoptosis (6). Pro-inflammatory cytokines, such as interleukin-1 (IL-1) and tumor necrosis factor-α (TNF-α), can induce expression of inducible NOS (iNOS) that generates larger quantities of NO, resulting in cardiac cell degeneration and apoptosis (7,8). The cytokine-induced expression of iNOS can occur in both ischemic and nonischemic heart failure, septic cardiomyopathy, cardiac allograft rejection, and myocarditis. Suppression of the cytokine-inducible, NO-synthesizing enzyme may reduce generation of cytotoxic reactive nitrogen radicals, leading to increased survival of grafted or pre-existing stem cells in the diseased hearts with inflammation.

Cholesterol Synthetic Pathway.

3-hydroxy-3-methyl glutaryl coenzyme A (HMG CoA)-reductase acts as the rate-limiting enzyme for endogenous cholesterol synthesis. The HMG-CoA reductase inhibitors, statins or vastatins, are widely used cholesterol-lowering medicines for both primary and secondary prevention of atherosclerosis or coronary heart disease. Statins can effectively diminish endogenous cholesterol synthesis and reduce the plasma LDL cholesterol levels in patients with hypercholesterolemia (9,10). It has been reported that treatment with statins benefits the coronary endothelium and contributes to plaque stabilization in patients with coronary artery disease (42). In addition, improved clinical outcomes after coronary stent implantation in patients undergoing statin therapy have been demonstrated (43). Those effects can be achieved either by anti-apoptotic mechanisms or by promoting endogenous repair mechanisms with accelerated re-endothelialization of damaged vessel tissues involving the mobilization and incorporation of bone marrow-derived endothelial progenitor cells (44). Other beneficial effects of statins on cardiac tissue and function have also been reported, which could be explained by the anti-apoptotic and prodifferentiating effects of statins on progenitor cells. Indeed, recent studies using animal models of myocardial ischemia or infarction (17,18,45) and human clinical trials (18,46) suggest the clinical relevance of HMG-CoA reductase inhibitors as potential therapeutic agents for the treatment of patients with myocardial infarction and heart failure. It has been previously demonstrated that treatment with statins may confer cardioprotection in isolated perfused hearts during ischemia-reperfusion, which is in part due to a reduction in myocyte apoptosis (17). Very recently, it has been shown that statin treatment protects embryonic myocyte progenitors against the cytotoxicity caused by cytokine-induced high output of nitric oxide (NO) production induced by inflammatory cytokines (45). The anti-inflammatory effect of statins has been documented in diseased cardiovascular tissues (47). The effects of statins on restoration of vascular functions and the promotion of endogenous repair by bone marrow-derived stem/progenitor cell recruitment have been also reported (44,39).

The molecular basis for the many diversified bioactivities of statins is not completely understood. Increasing evidence indicates that certain pharmacological effects of statins occur independently of cholesterol. Indeed, the mevalonate pathway can produce certain by-products or intermediates, in addition to cholesterol, that are essential for late fetal and early neonatal tissue development, namely isoprenoid compounds (48,49). Research has suggested that HMG-CoA reductase inhibitors may seriously alter the cholesterol homeostasis in the brain and therefore influence the normal growth, development, and survival of neurons (50). A caution has been raised about the adverse side-effects of long-term statin therapies on the central nervous system (51,52). However, other investigations have pointed to the potential benefits of statin treatment to increase survival and differentiation of oligodendrocyte progenitors in an animal model of multiple sclerosis (50) and to induce neuroglial differentiation of bone marrow-derived human mesenchymal stem cells (53). Moreover, statins have been shown to induce osteoblast differentiation of embryonic stem cells (ESCs) (54,55) and bone marrow-derived stem cells but not stem cell proliferation (56).

Recent studies suggest that by blocking the mevalonate pathway, statins exert pleiotropic effects on vascular cell function that may not be related directly to cholesterol synthesis (11,12). Accumulating evidence indicates the involvement of the intermediates or by-products, e.g. isopranoids, from the mevalonate-cholesterol synthesis pathway in regulation of intracellular signal transduction and activation of transcriptional factors critical for inflammatory proteins and iNOS gene expression in heart failure (13). However, there are controversial reports on the statin regulation of iNOS expression in different cell types. For instance, Pahan et al. (14,15) showed that lovastatin inhibits iNOS and cytokine expression in rat macrophages, astrocytes, and microglia via reducing Ras farnesylation and inactivating NF-κB inactivation. By contrast, Hattori et al. (16) reported that statins augment cytokine-mediated induction of NO synthesis in vascular smooth muscle cells through altering synthesis of tetrahydrobiopterin (BH4), a key co-factor for iNOS, with little influence on NF-κB activities. Recent work have shown that statin treatment may confer cardioprotection in the isolated-perfused hearts during ischemia-reperfusion, which is in part due to a reduction in myocyte apoptosis (17).

Synthesis of Non-Sterol Isoprenoids and Protein Prenylation.

The HMG-CoA reductase inhibitors statins are potent inhibitors of cholesterol biosynthesis. However, the overall clinical benefits observed with statin therapy appear to be greater than what might be expected from changes in the lipid profile alone, suggesting that the beneficial effects of statins may extend beyond their effects on serum cholesterol levels. Inhibition of HMG-CoA reductase by statins may exert pleiotropic effects on cellular signaling and cellular functions involved in inflammation. Statin-sensitive signaling molecules include Rho guanosine triphosphatases (GTPases), mitogen-activated protein kinases, and Akt (61); statin-sensitive cellular functions include adhesion, chemotaxis, and release of superoxide anion (O₂⁻) and cytokines. It is generally believed that the majority of the observed anti-inflammatory effect of inhibition of HMG-CoA by statins can be attributed to the reduction in the cellular levels of isoprenoids and the prenylation of signaling proteins (e.g., Rho GTPases) (62-66). Rapidly accumulating evidence indicates that statins exert regulatory effects on cellular signaling via inhibiting protein prenylation by depleting the cellular pool of isoprenoids (e.g., geranylgeranyl-pyrophosphate) downstream of mevalonic acid (the product of HMG CoA reductase), as various effects of statins on cellular signaling and functions can be blocked by co-incubation of cells with mevalonic acid or isoprenoids. One of the genes whose expression is regulation by the protein prenylation-associated signaling is nitric oxide synthase (NOS) (62), an enzyme catalyzes conversation of L-arginine to L-cirtrulline and nitric oxide, a gaseous signaling molecule. Protein prenylation mediated by isoprenoids from the mevalonate pathway has impacts on expression of at least two isoforms of NOS, eNOS (61,62,67) and iNOS (68,45), through regulation of the gene promoter activities.

Cholesterol Synthesis Essentialfor Embryonic Development.

Cholesterol synthesis and metabolism appear to be essential for normal embryonic development (69,70). Target knockout of the HMG-CoA reductase gene is lethal (71). The embryos homozygous for the Hmgcr mutant allele can be recovered at the blastocyst stage, but not at E8.5, indicating that HMG-CoA reductase is crucial for early development of the mouse embryo. Supplementing the dams with mevalonate partially rescues the lethal phenotype. The importance of cholesterol for embryonic development is also evidenced by the finding that the distal inhibitors of cholesterol synthesis are highly teratogenic in animals (72-74). These inhibitors of cholesterol synthesis include AY 9944 and BM 15766 that inhibit 7-dehydrocholesterol reductase, and triparanol that inhibits Δ²⁴-dehydrocholesterol reductase. Both the enzymes catalyze the last step of cholesterol synthesis. Treatment with these inhibitors caused holoprosencephalic brain anomalies, a severe genetic defect seen in patients with Smith-Lemli-Opitz syndrome (75,76,86), which is a recessive autosomal genetic disease characterized by malformations (microcephaly, corpus callosum agenesis, holoprosencephaly, and mental retardation), male pseudohermaphroditism, finger anomalies, and failure to thrive. The human genetic disease is another example of the genetic impact of cholesterol deficiency on embryonic development. Patients with this genetic disorder typically have a deficit in 7-dehydrocholesterol reductase, a severe hypocholesterolemia and an accumulation of precursors: 7-dehydrocholesterol, 8-dehydrocholesterol, and oxidized derivatives. The presence of 7-dehydrocholesterol in the serum of patients is pathognomonic. Administration of high concentrations of cholesterol can effectively improve the patient's clinical outcome. In the past few years, several distinct inherited disorders (77,78) have been linked to different enzyme defects in the cholesterol biosynthetic pathway, in addition to SLOS. Patients afflicted with these disorders are characterized by multiple morphogenic and congenital anomalies including internal organ, skeletal and/or skin abnormalities. By the finding of abnormally increased levels of intermediate metabolites, patients may be treated with normal products or transduced with normal genes that correct the disease-causing mutations in genes encoding the implicated enzymes.

Statins and Statin-Like Compounds

The term “statins” refers to a group of functionally and structurally similar compounds that inhibit the enzyme 3-hydroxy-3-methylglutaryl coenzyme A (HMG-CoA) reductase. Statins have a lactone structure or open lactone structure, as it known. Known statins include cerivastatin, marketed as Baycol® by Bayer (See U.S. Pat. Nos. 5,006,530 and 5,177,080), lovastatin, marketed as Mevacor® by Merck (See U.S. Pat. No. 4,963,538), simvastatin, marketed as Zocar®, pravastatin, marketed as Pravachol®, atorvastatin, marketed as Lipotor® by Warner-Lambert (See U.S. Pat. No. 5,273,995), fluvastatin, marketed as Lescol® (See U.S. Pat. No. 4,739,073), and rosuvastatin, marketed as Crestor® (See, e.g., WO 02/41895). Another statin is NK-104 developed by NEGMA (87). Additional compounds of similar structure and/or which inhibit enzymes of the isoprenoid/steroid pathway, such as HMG-CoA reductase, are termed “statin-like” compounds. The ability of a compound to inhibit these enzymes can be determined by standard assays well known in the art, and as described below.

Statin's Effect on Stem Cell Function

The HMG-CoA reductase inhibitor statins are the most widely described drugs that can effectively lower blood cholesterol levels, and prevent atherosclerosis and atherosclerosis-associated coronary heart disease. However, there has been a debate on whether statins exert teratogenic effects on embryonic development. Early reports (79-82) suggest that certain forms of statins may cause abnormalities in embryogenesis, including mevinolinic acid, Compactin and lovastatin. However, compared to the distal inhibitors of cholesterol synthesis, the statin effect is relatively minor. In fact, recent work on adult stem cells indicates that treatment with statins increases the number of circulating adult stem cells in patients with atherosclerosis. The statin-mediated HMG-CoA reductase inhibition has a biphasic dose-dependent effect on angiogenesis in a manner independent of cholesterol synthesis and associated with alterations in endothelial apoptosis and growth signaling. The non-sterol isoprenoid geranylgeranyl pyrophosphate may partially reverse the statin's effect, suggesting the involvement of geranylated proteins.

Statins also enhance new bone formation in vitro and in rodents. This effect is associated with increased expression of the bone morphogenetic protein (BMP) gene in bone-forming stem cells. Lovastatin and simvastatin can increase bone formation when injected subcutaneously over the calvaria of mice and increased cancellous bone volume when orally administered to rats (88). Thus, in appropriate doses, statins may have therapeutic applications for the treatment of osteoporosis. The mechanism by which statins induces BMP expression is unclear. There is evidence that lovastatin stimulates rapid activation of Ras, which associates with and activates PI 3 kinase in plasma membrane which in turn regulates Akt and Erk1/2 to induce BMP-2 expression for osteoblast differentiation (89). Interestingly, BMP expression is also closely associated with cardiac myogenesis. In 1997, Schultheiss T M, et al. (90) demonstrated that bone morphogenetic protein (BMP) signaling plays a central role in the induction of cardiac myogenesis in the chick embryo. At the time when chick precardiac cells become committed to the cardiac muscle lineage, they are in contact with tissues expressing BMP-2, BMP-4, and BMP-7. Application of BMP-2-soaked beads in vivo elicits ectopic expression of the cardiac transcription factors CNkx-2.5 and GATA-4. Furthermore, administration of soluble BMP-2 or BMP-4 to explant cultures induces full cardiac differentiation in stage 5 to 7 anterior medial mesoderm, a tissue that is normally not cardiogenic. The competence to undergo cardiogenesis in response to BMPs is restricted to mesoderm located in the anterior regions of gastrula- to neurula-stage embryos. The secreted protein noggin, which binds to BMPs and antagonizes BMP activity, completely inhibits differentiation of the precardiac mesoderm, indicating that BMP activity is required for myocardial differentiation in this tissue. Together, these data imply that a cardiogenic field exists in the anterior mesoderm and that localized expression of BMPs selects which cells within this field enter the cardiac myocyte lineage. Because statins and isoprenoid pathway inhibitors activate the BMP genes, they may exert effects on stem cells by promoting osteogenesis and myogenesis

### SUMMARY OF THE INVENTION

In accordance with certain embodiments of the present invention, a method is provided for enhancing survival and differentiation of stem cells and cells of myogenic lineage derived from the stem cells, when the cells are exposed to an inflammatory or apoptotic stimulus. This method includes culturing stem cells in vitro in a medium containing a statin, to produce statin-pretreated cells with enhanced resistance to inflammatory or apoptotic stimuli. In some embodiments, the statin-pretreated cells are allowed to differentiate in the presence of the statin, to produce statin-pretreated cells of myogenic lineage.

In some embodiments, the method further includes exposing the pretreated stem cells, or the statin-pretreated cells of myogenic lineage, to an inflammatory or apoptotic stimulus. In some embodiments, the statin-pretreated cells are exposed to the inflammatory or apoptotic stimulus in vitro, such as when testing a candidate statin or another compound for modulation of the above-described protective effect. For example, the method may include exposing the statin-pretreated cells to the inflammatory or apoptotic stimulus in vitro, and then determining whether survival and differentiation of statin-pretreated stem cells is enhanced compared to non-statin-pretreated stem cells, or statin-pretreated cells of myogenic lineage derived from the stem cells, which are likewise exposed to the stimulus.

In some embodiments, the statin is a statin or statin-like compound that inhibits HMG-CoA reductase. Such inhibitory compounds include, but are not limited to, atorvastatin, cerivastatin, fluvastatin, lovastatin, mevastatin, pitavastatin, rosuvastatin, simvistatin and ezetimibe and combinations of those.

In some embodiments, the statin pre-treated cells are either embryonic stem cells or adult stem cells residing in adult tissues, and in some embodiments, the cells of myogenic lineage comprise differentiated or undifferentiated cardiac or vascular myoblasts. For example, the statin pre-treated cells may be embryonic cardiac myoblasts or embryonic vascular myoblasts.

In some embodiments, an above-described method further includes transplanting into a diseased tissue or organ the statin pretreated stem cells, or statin-pretreated cells of myogenic lineage derived from the stem cells, in a medium comprising the statin or one or more isoprenoid pathway inhibitor. A diseased tissue or organ may be, for example, an ischemic or infarcted tissue or organ. In some embodiments, the statin and the isoprenoid pathway inhibitor(s) are both included in the medium.

In some embodiments, exposing the statin-pretreated cells to the inflammatory or apoptotic stimulus occurs in vivo, after implanting or transfusing into a host the statin-pretreated stem cells or the statin-pretreated cells of myogenic lineage derived from the stem cells. In certain embodiments, a statin is administered to the host prior to, or after, implantation or transfusion of the pretreated cells. The statin may be administered orally, intravenously or locally.

In some embodiments, the cells are cultured in the medium containing a statin for up to 24 hours, to produce the statin-pretreated cells. In certain embodiments, the medium contains the statin in a concentration of about 1 to 10 μg/mL. In some embodiments, an above-described method also includes exposing the statin-pretreated cells to at least one statin or at least one isoprenoid pathway inhibitor in vitro. In some embodiments, an above-described method includes locally administering the statin- or isoprenoid pathway inhibitor-pretreated cells in vivo.

Also provided in accordance with the present invention is a method of protecting implanted embryonic stem cells, or cells of myogenic lineage derived from the stem cells, from the cytotoxic effects of inflammatory or apoptotic stimuli. Such stimuli may occur when the tissue is exposed to one or more inflammatory or apoptotic agent, such as oxidized low density lipoprotein (oxLDL), oxysterols, cytokines and Fas ligand, for example. This method includes transplanting into a vascular or cardiac tissue of a mammal a plurality of in vitro statin-pretreated embryonic stem cells, or statin-pretreated cells of myogenic lineage derived from such stem cells, whereupon apoptosis in the transplanted statin-pretreated cells is inhibited. In some embodiments, prior to being transplanted, the stem cells are uncommitted and are capable of differentiating into cells having a vascular or cardiac myocyte phenotype when transplanted into the cardiac or vascular tissue. In some embodiments, the stem cells are autologous or allogenic to the mammal. In some embodiments, the vascular or cardiac tissue is in a mammal with heart failure or a myocardial infarction or at least one atherosclerotic lesion, such as an aneurism or is an unstable plaque caused by hyperlipidemia, for example.

In some embodiments of the aforesaid methods, the inhibition of apoptosis deters apoptotic cell death of the transplanted stem cells or myocytes.

In some embodiments, an above-described method includes contacting the statin-pretreated cells with a non-steroidal isoprenoid to modulate the effect of the statin. In some embodiments, a method may further include administering clusterin to the tissue. This could be done separately or with administration of at least one isoprenoid pathway inhibitor, to enhance protection of the statin-pretreated embryonic stem cells, or statin-pretreated cells of myogenic lineage derived from the stem cells, from cytotoxic effects of inflammatory or apoptotic stimuli.

In some embodiments, an aforesaid method comprises, prior to transplanting into a mammalian tissue, treating stem cells in a medium containing at least one statin, at least one bone morphogenic protein and clusterin, to promote myogenic differentiation of the stem cells. These and other features, advantages and embodiments will be apparent with reference to the following detailed description, drawings and claims.

### BRIEF SUMMARY OF THE DRAWINGS

FIG. 1 is a graph showing that simvastatin treatment reduces iNOS mRNA levels in TNF-α- and IL-1α-stimulated cardiac myoblasts. Reverse transcription-PCR for iNOS mRNA and 18S rRNA was performed using total RNA isolated from H9c2 cardiac myoblasts exposed to simvastatin (10⁻⁸ to 10⁻⁵ mol/L) in the presence of TNF-α (20 ng/mL) (closed squares) or IL-1α (20 ng/mL) (closed roots) for 24 h. PCR products were semi-quantified by densitometry after electrophoresis on agarose gels stained with ethidium bromide. The steady state level of iNOS mRNA was normalized to the level of the 18S rRNA against unstimulated controls. Data show mean ±S.D. from triplicate experiments.

FIG. 2 is a group of immunoblots and bar graphs showing that simvastatin reduces iNOS protein expression in TNF-α- or IL-1α-stimulated cardiac myoblasts. Immunoblotting with anti-iNOS antibody was conducted with total proteins extracted from H9c2 cells exposed to simvastatin (10⁻⁸ to 10⁻⁵ mol/L) in the presence of IL-1α (20 ng/mL) (Panels A and C) or TNF-α (20 ng/mL) (Panels B and D) for 24 h. Protein bands probed by antibody are quantified by densitometry (Panels C and D). Data represent the mean ±S.D. from three separate experiments. *, P<0.05.

FIG. 3 is a graph showing the time course of nitrite production in cardiac myoblasts treated with IL-1α alone or in combination with simvastatin or L-mevalonate. The logarithmic regression curves show the time-dependent nitrite production in H9c2 cardiac myoblasts exposed to IL-1α (20 ng/mL) in the presence (closed squares) and absence (closed roots) of simvastatin (10⁻⁶ mol/L), or pretreated with simvastatin in combination with L-mevalonate (10⁻⁴ mol/L, closed triangles) for 30 min prior to the addition of IL-1α. Data represent the means ±S.D. from three separate experiments. *, p<0.05

FIG. 4 is a group of immunoblots and bar graphs showing that L-mevalonate blocks the simvastatin inhibitory effect on TNF-α- and IL-1α-induced iNOS protein expression in cardiac myoblasts. Immunoblotting with anti-iNOS antibody using total proteins extracted from H9c2 cardiac myoblasts treated with simvastatin (10⁻⁸ to 10⁻⁵ mol/L) alone or in combination with L-mevalonate (10⁻⁴ mol/L) for 30 min prior to the addition of IL-1α (20 ng/mL, Panels A and C) or TNF-α (20 ng/mL, Panels B and D). Immunoreactive iNOS bands were quantified by densitometry. Data represent the mean ±S.D. from three separate experiments. *, P<0.05.

FIG. 5 is an immunoblot and bar graphs showing that GGPP attenuates the simvastatin inhibitory effect on IL-1α-induced iNOS expression and nitrite production in cardiac myoblasts. Panels A and B. Immunoblotting with anti-iNOS antibody was conducted with total proteins extracted from H9c2 cells treated with increasing concentrations of simvastatin (10⁻⁸ to 10⁻⁶ mol/L) alone or in combination with geranylgeranylpyrophosphate (GGPP, 10⁻⁶ mol/L) for 30 min, followed by stimulation with IL-1α (20 ng/mL) for 24 h. Immunoreactive iNOS bands were quantified by densitometry. Data represent means ±S.D. from three separate experiments. Panel C. Nitrite concentrations were determined by Griess reaction in the media of H9c2 cells under the same treatment shown in panels A and B. Data show means ±S.D. from three experiments. *, p<0.05.

FIG. 6 is a groups of bar graphs showing that Rho-associated kinase mediates simvastatin suppressive effect on iNOS expression in IL-1α-stimulated cardiac myoblasts. Panel A. Immunoblotting with anti-iNOS antibody in H9c2 cardiac myoblasts pretreated with Y-27632 (10⁻⁶ to 10⁻⁵ mol/L) alone or in combination with 10⁻⁶ mol/L simvastatin for 30 min, followed by the addition of IL-1α (20 ng/mL) for 24 h. Immunoreactive iNOS bands were quantified by densitometry. Each bar represents the mean ±S.D. from three separate experiments. *, p<0.05. Panel B. Nitrite production was determined by Griess reaction with the media of H9c2 cell cultures treated under the same conditions shown in panel A. Each bar represents the mean ±S.D. from five separate experiments. *, p<0.05. Panel C. Rho kinase assays for assessing substrate protein phosphorylation. After treatment with cytokine and simvastatin or Y-27632, cells were lyzed, centrifuged, and resulting supernatants collected for enzymatic assays. The Rho Kinase-mediated protein phosphorylation was quantified by spectrophotometry at 450 nm. Data were presented as means ±S.D. (n=5). *, p<0.05; **, p<0.01

FIG. 7 is an autoradiogram and a group of immunoblots and bar graphs showing that simvastatin inactivates NF-κB and prevents its p65/RelA subunit from nuclear translocation in cytokine-stimulated cardiac myoblasts. Panel A. Electrophoretic mobility gel shift assay was performed by mixing ³²P-oligonucleotide coding for the consensus sequence of NF-κB binding promoter with the nuclear proteins from H9c2 cells preincubated with 10⁻⁶ mol/L simvastatin for 8, 18 or 24 h, followed by incubation with 20 ng/mL IL-1α for 15 min. Specificity of the NF-κB-DNA complex formation was determined by competition with unlabeled, cold oligonucleotide and by supershift with anti-NF-κB antibody. The autoradiogram is a representative of three separate gel shift experiments for NF-κB. Panels B-G. Immunoblotting with antibody against p65/RelA subunit of NF-κB in the total cellular (Panels B and C) or nuclear proteins (Panels D and G) extracted from H9c2 cells stimulated with IL-1α after 24 h preincubation with simvastatin 10⁻⁶ mol/L in the presence or absence of L-mevalonate and Y-27632 (Panel C). Immunoreactive p65 bands were quantified by densitometry (Panels E, F and G). Data represent the means ±S.D. from three separate experiments. *, P<0.05.

FIG. 8 is an immunoblot and bar graph showing that simvastatin elevates the phosphorylated IκBα intracellular pool without subsequent degradation in IL-1α stimulated cardiac myoblasts. Immunoblotting with antibody against ser32 phosphorylated IκBα in total proteins extracted from IL-1α-induced H9c2 cells after 24 h preincubation with simvastatin (10⁻⁸-10⁻⁶ mol/L) (Panel A). Immunoreactive bands were quantified by densitometry (Panel B). Each bar represents the means ±S.D. from three separate experiments. *, p<0.05.

FIG. 9 is a schematic presentation of the signal transduction pathways by which statins regulate iNOS expression in embryonic cardiac myoblasts.

FIG. 10 is a group of photomicrographs of undifferentiated and differentiated human embryonic stem cells (hESCs). a, hESCs undifferentiated; b, embryoid body (EB) at day 26, arrow showing beating myogenic colonies; c, microvascular structure in EBs. D, a high power view of a tubing structure in EB. (Arrows).

FIG. 11 is a series of graphs showing edge-motion detection of mESC-derived contractile myogenic cells untreated (upper) or treated with isoproterenol (10 ng/ml) in the absence (middle) or presence (lower) of propanolol (25 ng/ml).

FIG. 12 is an immunoblot and bar graph showing immunoblotting for ubiquitinated proteins in murine and human ESC. A, immunoblotting for ubiquitin (upper panel) and b-actin (lower). B, relative abundance of ubiquitin-proteins in murine and human ESC.

FIG. 13 is a pair of photomicrographs showing hESC-derived EB after treatment with MG132 for 2 days. a, untreated and b, treated.

FIG. 14 is a pair of HPLC radiochromatograms of [³H]-7-ketocholesterol and [³H]-cholesterol in human 293 embryonic kidney cells transfected with apolipoprotein-J. a, cellular lipids; and b, medial lipids. The cells were incubated with [³H]-7-ketocholesterol (0.5 uCi/ml) for 24 hrs. Lipids were extracted and analyzed by HPLC in both cells and media. Upper panel, cell lipids; Lower, media.

FIG. 15 is a graph showing TUNEL staining of mouse peritoneal macrophages. Macrophages loaded with acLDL were treated with CP-113,818 (▪), CP-113,818 plus U18666A (♦), or U18666A in medium containing 0.2% BSA (□). After treatment the cells were fixed and nuclear DNA fragments were labeled using a fluorescent TUNEL assay.

FIG. 16 is a graph showing statin Inhibition of iNOS expression in Embryonic Cardiac Myoblasts Exposed to Proinflammatory Cytokines for 24 hrs.

FIG. 17 is shows A, Western Blot for iNOS in embryonic cardiac myoblasts stimulated with IL-1α (20 ng/ML) in the presence or absence of simvastatin or simvastatin +/− geranylageranyl pyrophosphate (GGPP, 10⁻⁶ mol/L) for 24 hrs. A, Western blot with anti-iNOS and B, shows the densitometry of iNOS protein bands. N=3, mean ±SD.

FIG. 18 shows gel shift assays for the NF-kB activity. Nuclear proteins were extracted from cytokine-treated and untreated cardiac myoblasts in the presence or absence of simvastatin, and mixed with ³²P-NF-kB consensus or control double strand oligos. Protein-oligo complexes were detected by autoradiogram following electrophoresis.

FIG. 19 is a Western blot for phosphorylated IκBα in IL-1 stimulated cardiac myoblasts in the presence or absence of simvastatin. Upper panel shows densitometry of protein bands. N=3, mean ±SD.*, p<0.05.

FIG. 20 is a Western blot for cardiac sarcomeric α-actinin in simvastatin-treated murine ESCs. Total proteins extracted from simvastatin-treated murine ESCs. Western blot was conducted with anti-α-actinin and densitometry performed for semiquantitation of the protein levels, normalized by β-tubulin bands. N=4, mean ±SD; *, p<0.05.

FIG. 21 is a bar graph showing the development of beating myocytes in simvastatin (10⁻⁶ mol/L)-treated and untreated murine ESCs in the presence or absence of L-mevalonate (10⁻⁵ mol/L) for 8 days. Phase-contrast microscopy was used to quantify the region with beating myocytes.

FIG. 22 are bar graphs showing that simvastatin treatment enhances cardiac differentiation of murine embryonic stem cells. Embryoid bodies (EBs) in the hanging-drop cultures of ESCs were plated in duplicate in 6-well plates with 10 EBs per well and then treated with simvastatin with or without mevalonate. At day 12, the number of contracting EB outgrowths (Panel A) and extension of beating area (Panel B) were detrmined by morphometry. The results were plotted as percentages of the total EBs counted in each well and shown as the mean ±SD for each of the 3 separate experiments. *, P<0.05 vs untreated control; #, P<0.05 vs simvastatin-treated control.

FIG. 23 are immunoblots showing that simvastatin treatment enhances the expression of the cardiac-specific proteins α-actinin and myocardin A but not endothelial protein Tie-2 in murine embryonic stem cells. Immunoblot analysis of proteins from EBs treated with or without Simvastatin, was carried out with antibodies against cardiac-specific proteins, such as sarcomeric α-actinin (Panels A and D) and myocardin A (Panels B and E), as well as endothelial-specific proteins such as Tie-2 (Panels C and F). β-Tubulin staining of stripped blots was used as a control. Intensity of immunoreactive protein bands was assessed by densitometry (Panels D-F). *, P<0.05 vs untreated cells.

FIG. 24 is a series of immunoblots showing expression of Bad, Bcl-x_{L}, and PCNA in embryonic myoblasts treated with or without simvastatin in the presence or absence of IL-1α. Expression of Bad (BD Biosciences, San Jose, Calif.), Bcl-x_{L} (BD Biosciences), and PCNA (BD Biosciences) in H9C2 embryonic myoblasts stimulated with IL-1 in the presence or absence of simvastatin, was analyzed by immunoblotting with monoclonal antibodies against Bad (Panel A, upper), Bcl-x_{L} (Panel B, upper), and PCNA (Panel C, upper). Quantitation of protein bands was achieved by densitometry (Panels D, E and F, bottom). β-Tubulin staining of stripped blots was used as a control. Data show the mean ±SD for each of the 3 separate experiments. #, P<0.05 vs untreated cells; *, P<0.05 vs IL-1-treated cells; **, P<0.05 vs IL-1+simvastatin-treated cells. PCNA, proliferating cell nuclear antigen; IL-1, interleukin-1-α; iNOS, inducible nitric oxide synthase; MnSOD, manganese superoxide dismutase.

FIG. 25 is an immunoblot assay showing the expression of iNOS and MnSOD in murine embryonic stem cells treated with or without Simvastatin. Expression of iNOS (Panels A and C) and MnSOD (Panels B and D) in murine ESCs in the presence or absence of simvastatin was analyzed by immunoblotting. Quantitation of protein bands was achieved by densitometry (panels C and D). β-Tubulin staining of stripped blots was used as a control. Data show the mean ±SD for each of the 3 separate experiments. #, P<0.05 vs untreated cells.

FIG. 26 is a group of photomicrographs showing that simvastatin prevents IL-1-induced apoptosis in H9c2 embryonic myoblasts. The cells were exposed with IL-1αSimvastatin for 24 hours and then stained with two fluorochromes: acridine orange and ethidium bromide. Living (green fluorescence) and apoptotic (red fluorescence) cells were identified and counted with an Olympus fluorescence microscope connected to a computer imaging station. A, untreated; B, IL-1; C-E, IL-1+simvastatin at different concentrations; F, IL-1+simvastatin+mevalonate 10⁻⁴ mol/l. Sim, simvastatin; Mev, mevalonate; IL-1, interleukin-1α.

### DETAILED DESCRIPTION

It is now proposed that at least some statins and isoprenoid pathway inhibitors can be advantageously used to influence cardiovascular stem cell and/or myocyte survival, proliferation and differentiation. Despite the recent knowledge that statin treatment may confer cardioprotection in isolated perfused hearts during ischemia-reperfusion, the molecular mechanism underlying the statin protective effect is unknown. Because stem cells participate in post-ischemic heart repair, it was of interest to determine whether statin treatment inhibits cytokine-induced expression of iNOS in premature, undifferentiated cardiac myoblasts. In this study, a cell culture system was employed to determine whether inhibition of HMG-CoA reductase by simvastatin alters expression of iNOS in embryonic, undifferentiated cardiac myoblasts exposed to proinflammatory cytokines. The resulting data showed that simvastatin treatment significantly reduced expression of iNOS in cardiac myoblasts stimulated with cytokines. The statin inhibitory effect might occur through a mechanism in which isoprenoids, an intermediate or similar by-products from cholesterol synthesis, regulate activation of several key intracellular signalling proteins, such as Rho A kinase and NF-κB. Taken together, this data strongly supports the notion that statins have regulatory effects on iNOS expression by undifferentiated myoblasts during the development of cardiac failure and inflammation. The present investigation also included determining whether a statin (e.g., simvastatin), at therapeutic doses, would affect embryonic stem cell (ESC) myogenic differentiation and resistance to apoptosis. The impact of endogenous cholesterol depletion by inhibition of HMG-CoA reductase was also determined. It was found that simvastatin, at therapeutic doses, promotes ESC myogenic differentiation and increased their resistance to apoptosis.

Materials and Methods

Materials. Human recombinant interleukin(IL)-1α and tumor necrosis factor(TNF)-α were from R&D Systems Inc. (Minneapolis, Minn.), L-mevalonate was obtained from Sigma, geranylgeranylpyrophosphate (GGPP) and farnesylpyrophosphate (FPP) were from Biomol Research Laboratories Inc. (Plymouth, Pa.), Rho inhibitor Y-27632 was from Calbiochem (S. Diego, Calif.). Simvastatin obtained from Merk Sharp & Dohme (Rome, Italy) was activated to its active form by alkaline hydrolysis before use. Briefly, 4 mg of simvastatin prodrug were dissolved in 8 mL of NaOH 0.1 N/NaCl 0.154 mol/L solution, and then incubated at 50° C. for 2 h. The pH was brought to 7.0 by HCl. The final concentration of the stock solution adjusted to 4 mg/mL and stored at −20° C. (45).

Cell cultures. H9c2 cells purchased from American Type Culture Collection (ATCC, Rockville, Md.) are spontaneously immortalized ventricular myoblasts from the rat embryo, with preservation of several electrical and biochemical characteristics found in adult cardiomyocytes. They were cultured in DMEM medium (ATCC) supplemented with 10% heat-inactivated fetal bovine serum (FBS) in 95% air and 5% CO₂ at 37° C. At subconfluence (70-80%), H9c2 myoblasts cultured at petri dishes or 24-well plates were pre-exposed to simvastatin and then stimulated with the proinflammatory cytokine, IL-1-α or TNFα, in the presence or absence of isoprenoids, the intermediates or by-product from cholesterol synthesis, or mevalonate (10⁻⁴ mol/L) (45). After certain time intervals, 100 μL/well culture media were collected for determination of nitrite production.

The murine ESC (mESC) line CCE was obtained from American Type Culture Collection (ATCC, Rockville, Md.). Murine ESCs were cultured without feeder cells in Dulbecco's modified Eagle's medium supplemented with 10% ESC-qualified Fetal Bovine Serum (FBS) (Stem Cell Technologies, Vancouver, Va.), pyruvate (Stem Cell Technologies, stock solution diluted 1:100), 2 mM L-glutamine, nonessential amino acids (Stem Cell Technologies, stock solution diluted 1:100), 100 IU/mL penicillin, 0.1 mg/mL streptomycin, and leukemia inhibitory factor (LIF) (Stem Cell Technologies). To induce differentiation, EBs were formed from undifferentiated mESCs in hanging drops of 400 cells in 20 μL of medium without LIF. After 5 days in suspension, EBs were plated on gelatin-coated dishes and cultured in cardiomyocyte-differentiation medium (Iscove's modified Eagle's medium, supplemented with 15% FBS, 2 mM L-glutamine, 5×10⁻⁵ M β-mercaptoethanol, nonessential aminoacids, 100 IU/mL penicillin, 0.1 mg/mL streptomycin).

Statin Treatment. At plating on day zero, EBs were treated with simvastatin (10⁻¹-10⁻⁶ mol/L), which blocks HMG-CoA reductase in the presence or absence of mevalonate (10⁻⁴ mol/L). The inhibition of endogenous cholesterol synthesis was the mechanism used for analyzing the effect of endogenous cholesterol depletion on EB differentiation. By days 8 through 12 after plating, spontaneously contracting cell clusters could be observed within the EB outgrowths. Culture media from both simvastatin-treated and untreated cells were changed each day after day 3.

The rat embryonic myogenic cell line H9c2 was commercially obtained from ATCC and maintained in Dulbecco's modified Eagle's medium (GIBCO) supplemented with 10% FBS at 37° C. in 95% air and 5% CO₂. At subconfluence (70%-80%), H9c2 myoblasts cultured in petri dishes or 24-well plates were pre-exposed to simvastatin (10⁻⁸-10⁻⁶ mol/L) and then stimulated with the proinflammatory cytokine IL-1-α (20 ng/mL) for 24 hours in the presence or absence of mevalonate (10⁻⁴ mol/L) (45).

Assay for NO Production. The activity of iNOS was determined in the culture medium by assaying nitrites, taken as an index of NO production, using the Griess reagent (1% sulphanilic acid and 0.1% N-[1-naphtyl] ethylenediamine-HCl in 5% phosphoric acid) as reported previously (4,18,19). Equal volumes of medium and Griess reagent were mixed, and the purple products quantified spectrophotometrically at 550 nm. Nitrite concentrations were determined from a linear standard curve constructed with known concentrations of sodium nitrite (0 to 40 μmol/L nitrite).

Immunoblotting. Total proteins were isolated from rat cardiac myoblasts (H9c2 cells) or embryonic stem cells in an ice-cold lysis buffer containing 10 mmol/L Tris (tris(hydroxymethyl)aminomethane) (pH 7.4), 1% sodium dodecyl sulfate (SDS) and 1× protease inhibitor (1 mmol/L sodium orthovanadate). Proteins (15 μg/lane) were separated under the reducing conditions (125 mM Tris pH 6.8, 4% SDS, 10% glycerol, 0.006% bromophenol blue, 2% β-mercaptoethanol) by electrophoresis onto 5-10% SDS-polyacrylamide gel and electro-blotted to nitrocellulose membranes (Osmonics, Westborough, Mass.). The membranes were reversibly stained with Ponceau red (Sigma) to verify equal protein loading and/or transfer. After blocking in Tris-buffered saline (0.2 M Tris and 8% NaCl) containing 5% non-fat powdered milk and 0.1% Tween 20 for 1 hour at room temperature, the membranes were incubated overnight at 4° C. with following primary antibodies to (a) iNOS (Transduction Laboratories, Lexington, Ky.; BD Biosciences); (b) NF-κB p65/rel (Santa Cruz Biotechnologies, Santa Cruz, Calif.); (c) ser³²-phosphorylated inhibitor IκBα (Santa Cruz); (d) Bad (BD Biosciences, San Jose, Calif.); (e) Bcl-x_{L} (BD Biosciences); (f) proliferating cell nuclear antigen (PCNA; BC Biosciences); (g) ryanodine receptor (Santa Cruze Biotechnology, Inc., Santa Cruz, Calif.); (h) α-sarcomeric actinin (Sigma); (i) myocardin A (developed by immunizing rabbits with a synthetic myocardin A peptide); (j) Tie-2 (Santa Cruz); (k) manganese superoxide dismutase (Santa Cruz); and (1) β-tubulin (Sigma). The blots were incubated with horseradish peroxidase-coupled secondary antibodies, washed and developed by using a SuperSignal West Pico Chemiluminescent Substrate Kit (Pierce, Rockford, Ill.). Intensity of each immunoreactive protein band was quantified by densitometric analysis. Cytokine-stimulated RAW 264.7 cells (Transduction Laboratories, Lexington, Ky.) were used as positive controls.

RNA isolation and RT-PCR. Total cellular RNA was isolated by a single extraction using an acid guanidinium thiocyanate-phenol-chloroform method with modification as reported elsewhere (17). Semi-quantitative multiplex reverse-transcription polymerase chain reaction (RT-PCR) was performed with a set of specific primers for iNOS. As the “house-keeping” controls, the 18S rRNA was also analysed by RT-PCR. The PCR products were visualized and quantified using a computerized densitometric system (BioRad Gel Doc 1000, Milan, Italy).

Electrophoretic mobility gel shift assay. Nuclear proteins were extracted from stimulated or unstimulated H9c2 cells. Cells were harvested and homogenized with an Ultra-Turrax T25-tissue homogenizer (Janke and Kunkel) in a low salt solution (0.6% Nonidet P-40 [NP-40], 150 mM NaCl, 10 mM HEPES pH 7.9, 1 mM EDTA, 0.5 mM phenylmethylsulfonyl fluoride [PMSF]), and centrifuged for 30 sec at 2000 rpm. The supernatant was incubated for 5 min on ice and then centrifuged for 5 min at 5000 rpm. The nuclei were resuspended in a high salt solution (25% glycerol, 20 mM HEPES Ph 7.9, 420 mM NaCl, 1.2 mM MgCl₂, 0.2 mM EDTA, 0.5 mM DTT, 0.5 mM PMSF, 2 mM benzamidine, 5 g/mL of each aprotinin, leupeptin and pepstatin). Protein concentrations were determined by Mio-Rad Laboratories Protein Assay (Bio-Rad Laboratories Inc, Hercules, Calif., USA). Double-stranded synthetic oligonucleotides NFκB motif (5′-AGT TGA GGG GAC TTT CCC AGG C-3′ and 5′-CCT GGG AAA GTC CCC TCA ACT-3′) was labeled with [γ-³²P]-ATP. Binding reactions containing 10 μg of crude nuclear extract were performed using the electrophoretic mobility gel shift assay core system (Promega, Madison Wis.) according to the manufacturer's protocol. For supershift experiments, mouse monoclonal anti-NF-κB antibody (1-2 μg) was added into the reaction.

Rho kinase assay. Cell pellets were homogenized in lysis buffer (50 mM NaCl, 50 mmol/L Tris-Hcl pH 8.0, 0.1% Triton X-100, 0.5 mM EDTA, 1 mM EGTA, 1 μg/ml pepstatin, 1 μg/ml leupeptin, 2 mM NaF, 2 mM sodium orthovanadate, 5 mM β-mercaptoethanol). After the cell lysates were centrifuged at 30.000 g for 30 minutes, supernatants were collected and incubated (100 μl/well) in 96-well plates pre-coated with a substrate corresponding to the myosin-binding subunit of myosin phosphatase, which contains a threonin residue selectively phosphorylated by Rho-kinase. Plates were incubated with HRP conjugated antibody specific for phosphorylated proteins, and then incubated with the HRP-substrate yielding colored products, which were subsequently quantified by spectrophotomerty at 450 nm. Purified Rho kinase was used as the positive controls and the cell lysates from the cultures treated with the Rho kinase inhibitor Y27632 as the negative controls.

Assays for Apoptosis. Analysis of apoptotic cells was performed by fluorescent microscopy with fluorochromes acridine orange and ethidium bromide (Sigma), as described previously (19). Apoptotic cells were discernible by their condensed, fragmented nuclei stained with both fluorochromes and were counted under a fluorescence microscope (Olympus, Center Valley, Pa.) connected to a computer imaging analysis station.

Nitrite Assays. Nitrite, a stable end-product of nitric oxide, was measured by using Griess reagent (1% sulphanilic acid and 0.1% N-[1-naphtyl] ethylenediamine-HCl in 5% phosphoric acid). Equal volumes of medium and Griess reagent were mixed, and the resulting purple product quantified by spectrophotometric assay at 550 nm. Nitrite concentrations were calculated from a linear standard curve constructed with known concentrations of sodium nitrite (0-14 μmol/L nitrite) (19).

Statistical Analysis. Two-group comparisons were performed by the Student's t-test for unpaired values. Comparisons of means of multiple groups were performed by analysis of variance (ANOVA), and the existence of individual differences, in case of significant F values at ANOVA, tested by Scheffé's multiple contrasts.

Transplantation of stem cells. In an animal model, statin-pretreated stem cells or myocytes are transplanted into a heart with or without infarction, using any suitable technique that is known in the art. It can then be determined whether the statin-pretreated stem cells or myocytes differentiate and/or survive better in an environment with inflammatory stimuli. For comparison, non-statin-pretreated stem cells or myocytes are similarly transplanted into a heart with or without infarction.

Echocardiography and ECG and Patch-clamp studies. After transplantation of the stem cells, morphological and functional changes are monitored using echocardiography using any suitable method which is known in the art, such as those described in U.S. patent application Ser. No. 11/252,260, the disclosure of which is hereby incorporated herein by reference. For example, in an animal model, 2D and M-Mode echocardiography may be performed after transplantation, e.g., at one, two, and four weeks. Electrophysiological changes are important features of cardiac dysfunction during myocardial infarction or ischemic heart failure. To characterize the electrophysiological alterations in the infarcted heart with statin-pretreated stem cell transplantation, any suitable methods may be used. Two such approaches are (1) in vivo study with electrocardiogram (ECG), and (2) in vitro study with the patch-clamp technique to measure ion channel functions.

Experimental Procedures

1. Simvastatin Inhibits iNOS Expression and Nitrite Production in Cardiac Myoblasts Induced by Proinflammatory Cytokines.

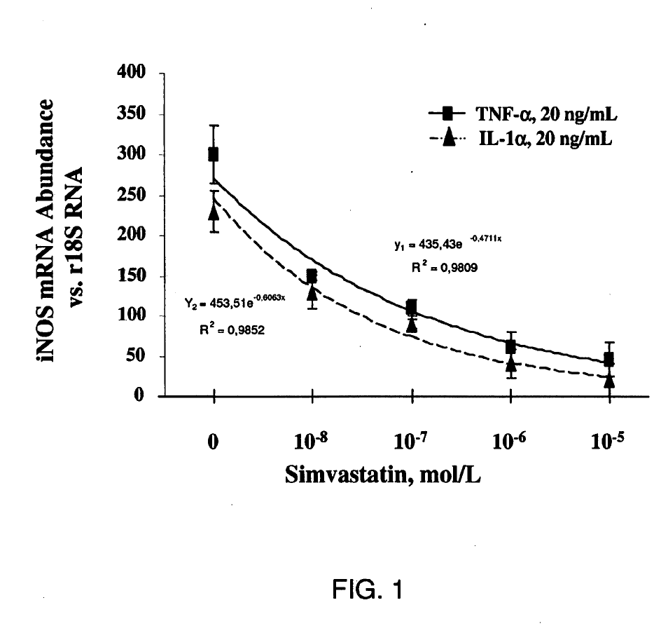

Exposure to IL-1α (20 ng/mL) or TNF-α (20 ng/mL) significantly increased the levels of iNOS mRNA in H9c2 cardiac myoblasts (FIG. 1), when compared those in normal, untreated H9c2 cells. In order to determine whether statins affect iNOS expression in embryonic cardiac cells, simvastatin was added into the cell cultures simultaneously with the cytokines. It was observed that in a concentration-dependent fashion, simvastatin markedly diminished expression of iNOS mRNA in H9c2 cells stimulated with the proinflammatory cytokines (FIG. 1). Under the same concentrations, both IL-1α and TNF-α stimulated cells showed a similar response to simvastatin in terms of iNOS mRNA expression. In the presence of simvastatin, IL-1α treated cells showed a dose-dependent decline in iNOS mRNA to the same or similar degrees as that in TNF-α treated cells (FIG. 1). Thus, simvastatin reduced steady-state levels of iNOS mRNA in H9c2 cardiac myoblasts stimulated with the proinflammatory cytokines.

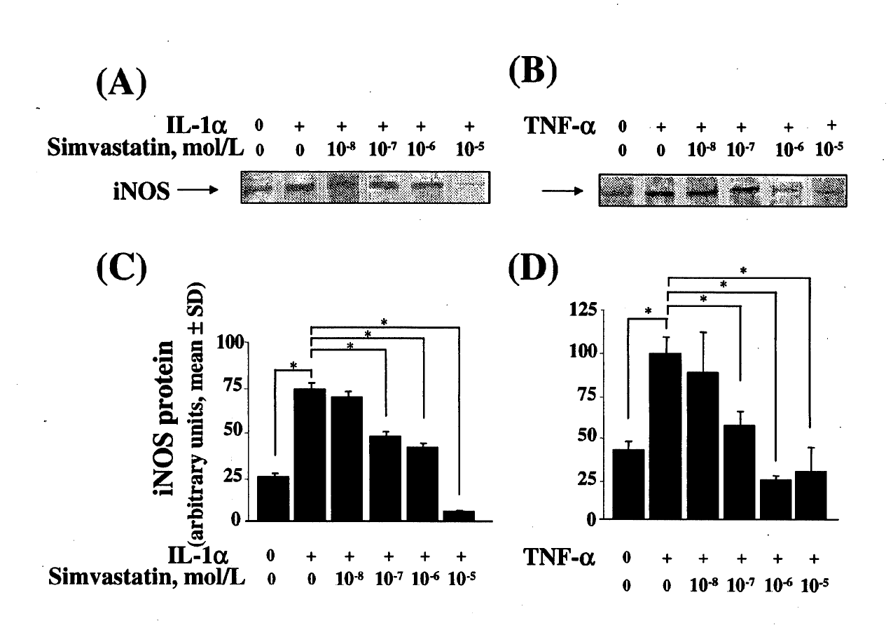

Further analysis of iNOS protein expression by immunoblotting with antibodies against iNOS confirmed the presence of high levels of iNOS expression in H9c2 cardiac myoblasts stimulated with IL-1α and TNF-α. Intense protein bands immunoreactive to anti-iNOS antibody corresponding to a molecular mass of 130 KDa were detected in the cytokine-stimulated H9c2 cells (FIGS. 2A-B). At the same concentrations, both the proinflammatory cytokines exerted stimulatory effects on iNOS protein expression in the cells. Interestingly, consistent to its effect on iNOS mRNA expression, simvastatin (10⁻⁸-10⁻⁵ mol/L) reduced expression of iNOS protein in H9c2 cells stimulated with IL-1α (FIGS. 2A and C) as well as TNF-α (FIGS. 2B and D). Thus, treatment with simvastatin inhibited expression of both iNOS mRNA and protein in cardiac myoblasts induced by proinflammatory cytokines.

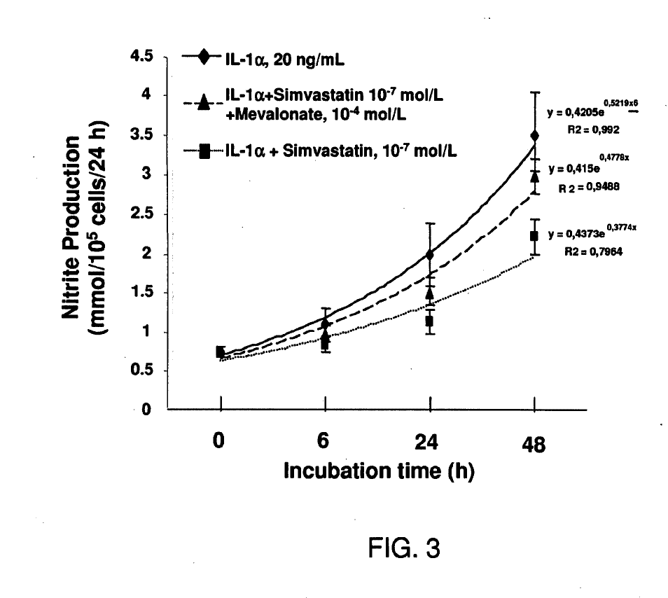

In order to further verify the statin inhibitory effect on iNOS gene expression, the accumulation of nitrite, a stable NO end-product reflecting the NOS activities, in the cultures treated with or without IL-1α and simvastatin was examined. Under the baseline condition without cytokine stimulation, H9c2 cells generated nitrite at the rates of approximately 0.8 μmol/10⁵ cells/24 h (FIG. 3). Stimulation with IL-1α (20 ng/mL) markedly increased nitrite production. The rates of nitrite production were more than 3.5 μmol/10⁵ cells/24 h in the cytokine-stimulated cells for the period of incubation from 24 to 48 h (p<0.05). Simvastatin (up to 10⁻⁶ mol/L) significantly diminished the cytokine-induced nitrite production in H9c2 cells (FIG. 3). The range of simvastatin concentrations leading to inhibition of nitrite production in cytokine-stimulated cells is comparable to the plasma levels of the drug in patients treated (20). Thus, at the pharmacological doses, simvastatin markedly reduced production of nitrite by H9c2 cells stimulated with the pro-inflammatory cytokines.

2. L-Mevalonate Mediates the Simvastatin Inhibitory Effect on iNOS Expression in Cardiac Myoblasts.

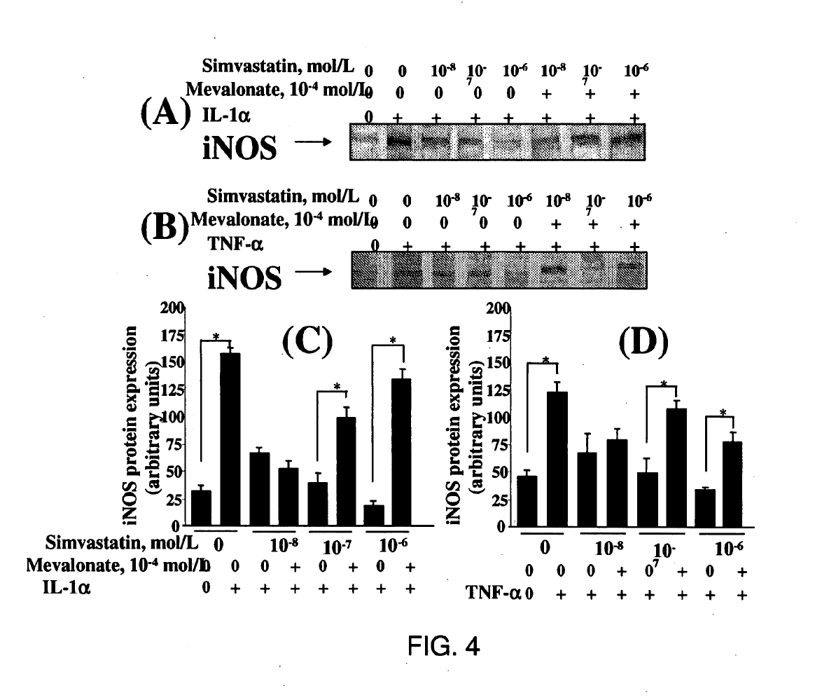

Generated by HMG CoA reductase, L-mevalonate serves as a key intermediate of cholesterol synthesis from acetyl-CoA. In order to determine whether the statin inhibition of iNOS gene expression occurred through blocking the HMG CoA reductase activity or reduction in L-mevalonate synthesis, exogenous L-mevalonate was added into the cultures of H9c2 cells with IL-1α (20 ng/mL) in the presence or absence of simvastatin (10⁻⁶ mol/L). It was observed that addition of L-mevalonate in excess amounts significantly increased the nitrite concentrations in the cultures of H9c2 cells exposed to a combination of IL-1α and simvastatin (FIG. 3). Examination of iNOS protein by immunoblotting revealed that L-mevalonate diminished the inhibitory effect of simvastatin by increasing iNOS protein expression in H9c2 cells treated with simvastatin plus IL-1α (FIGS. 4A and C) or TNF-α (FIGS. 4B and D). In the absence of simvastatin, L-mevalonate had no or little effect on cytokine-induced iNOS expression in H9c2 cells (FIG. 4), suggesting that L-mevalonate selectively blocked the simvastatin inhibitory effect on cytokine induction of iNOS in cardiac myoblasts. Because there was no major difference in induction of iNOS expression between IL-1α and TNF-α treated cells, the following experiments with simvastatin were mainly performed on the cells stimulated with IL-1α.

3. The Isoprenoid Intermediate GGPP, but not FPP, Blocks Simvastatin Inhibitory Effect on iNOS Expression.

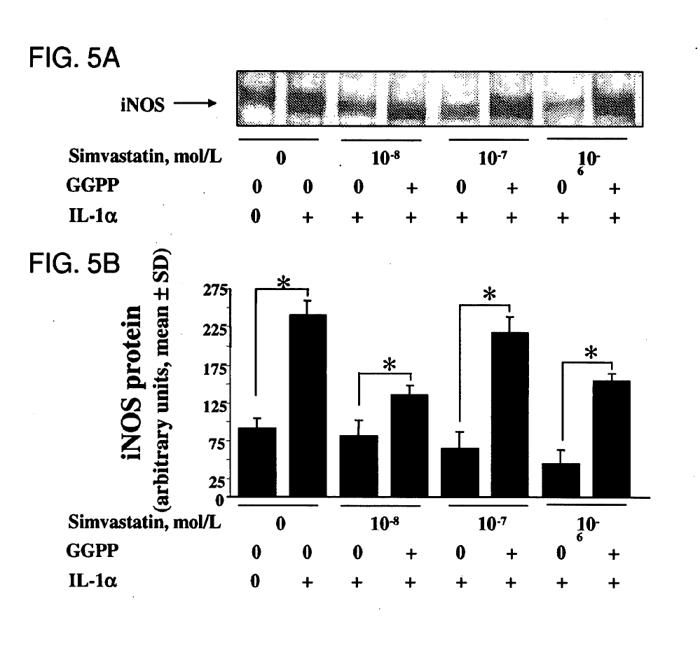

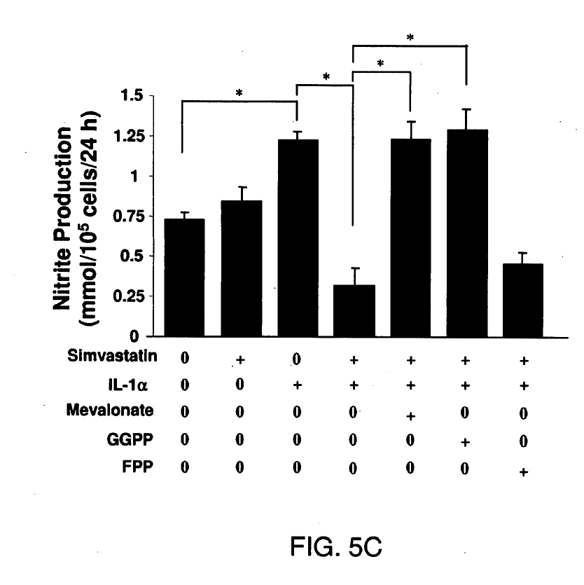

In addition to L-mevalonate, several downstream intermediates or by-products from the cholesterol biosynthetic pathway (21) may contribute to the inhibitory effect of simvastatin on expression of iNOS protein and activities. To exhaust the endogenous intermediates and by-products from cholesterol synthesis, H9c2 cells were pretreated with simvastatin (10⁻⁸ to 10⁻⁷ mol/L) up to 24 h, and then stimulated the cells with IL-1α (20 ng/mL) in the presence or absence of the isoprenoid intermediates, such as GGPP and FPP. Immunoblotting showed that addition of GGPP (10⁻⁷ to 10⁻⁵ mol/L) reversed the simvastatin-mediated suppression of the cytokine-induced expression of iNOS protein (FIG. 5A-B) and nitrite production (FIG. 5C) at almost the same levels to those in the L-mevalonate treated cells. In contrast, however, treatment with FPP at the same concentrations as GGPP did not alter nitrite accumulation in the cells pretreated with simvastatin and then IL-1α (FIG. 5C). These results suggested that GGPP but not FPP directly appeared to reverse the statin-induced inhibition of iNOS expression in the cardiac myoblasts stimulated with IL-1α.

4. Rho Kinase Contributes to Statin Inhibitory Effect on iNOS Expression in Cardiac Myoblasts Treated with IL-1α.

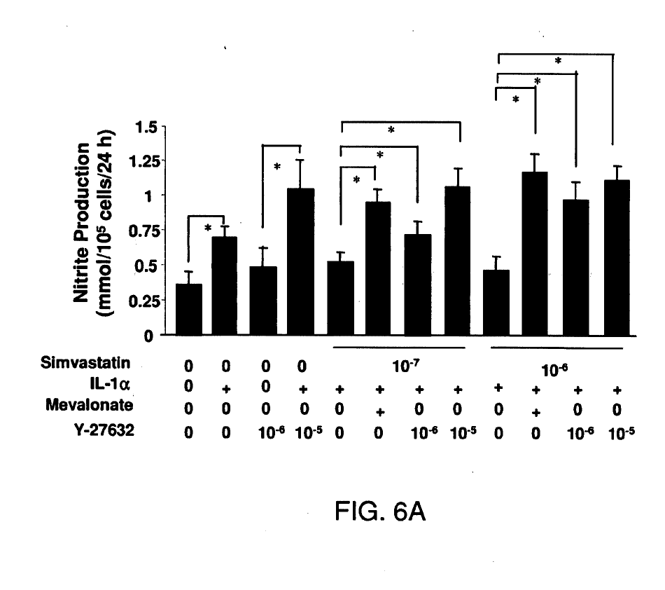

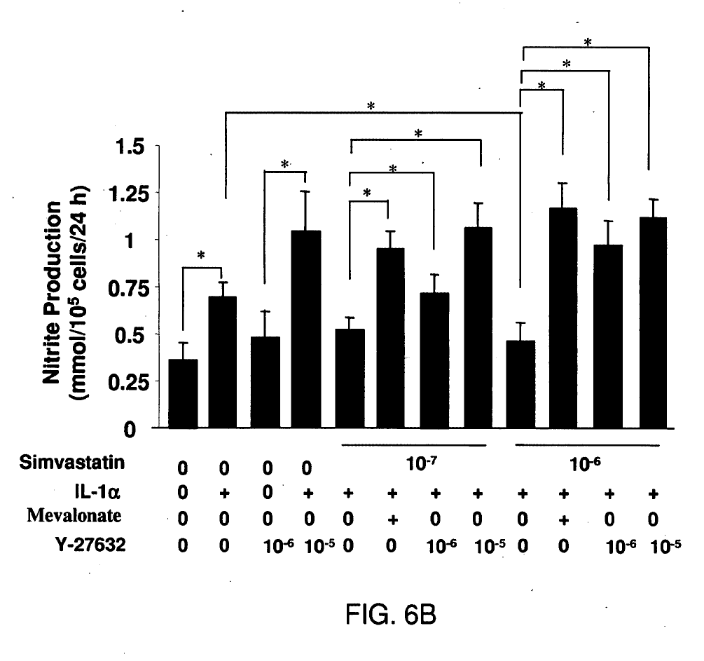

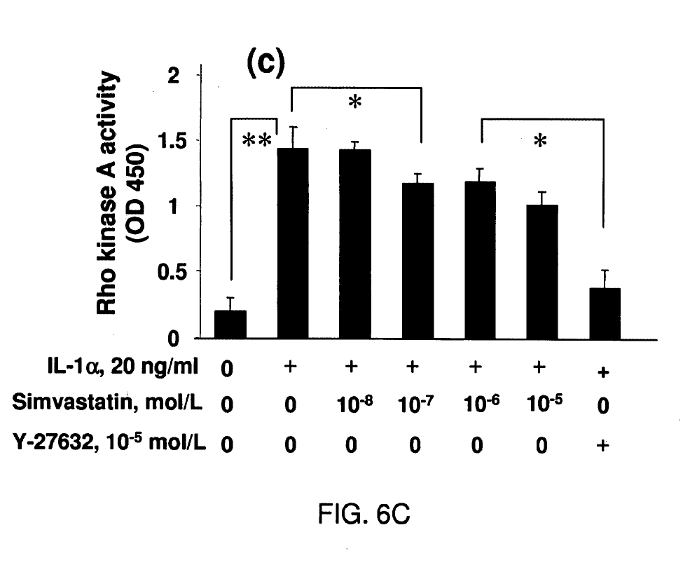

The Rho family of small GTP-binding proteins consists of three subfamilies, Rho, Rac and Cdc42, which play important roles in signal transduction and cell cycle regulation (22,23). The intermediate derivative of L-mevalonate, GGPP, acts as a lipid attachment to the Rho proteins, while FPP mainly targets Ras. Therefore, it was investigated whether the Rho kinase mediates the simvastatin effect on iNOS expression. The Rho kinase inhibitor Y-27632 (24) specifically inactivates p160ROCK, a key subunit of this kinase, known to regulate NO synthesis (11). It was tested whether Y-27632 affects iNOS expression in IL-1 treated H9c2 cells. It was observed that inhibition of Rho kinase with Y-27632 (10⁻⁶ to 10⁻⁵ mol/L) did not alter cytokine-induced iNOS protein expression in H9c2 cells (FIG. 6A). However, the Rho kinase inhibitor almost abolished the inhibitory effect of simvastatin on iNOS protein expression (FIG. 6A). Assessment of nitrite production indicated that Y-27632 dose-dependently increased the iNOS activity in the cytokine-treated H9c2 cells, suggesting the involvement of post-translational modification by Rho kinase in regulation of iNOS activities. Interestingly, in the presence of Y-27632, simvastatin treatment reduced neither NOS protein expression (FIG. 6A) nor nitrite accumulation (FIG. 6B). The reversal of the statin-induced nitrite reduction by Y-27632 occurred to the same extents as seen in the cells treated with L-mevalonate (FIG. 6B). To further confirm that Rho kinase A participates in the statin suppression of iNOS expression, the Rho kinase A bioactivities in H9c2 cells treated with both IL-1α and simvastatin was testedf by using enzymatic assays. It was found that simvastatin dose-dependently reduced Rho mediated protein phosphorylation (FIG. 6C), although the inhibitory effect appeared less striking than that of Y-27632. Thus, Rho kinase activation might be involved in the downstream events for simvastatin inhibition of iNOS expression.

5. Simvastatin Inactivates NF-κB through Elevation of Intracellular IκB in Cardiac Myoblasts Stimulated with Cytokines.

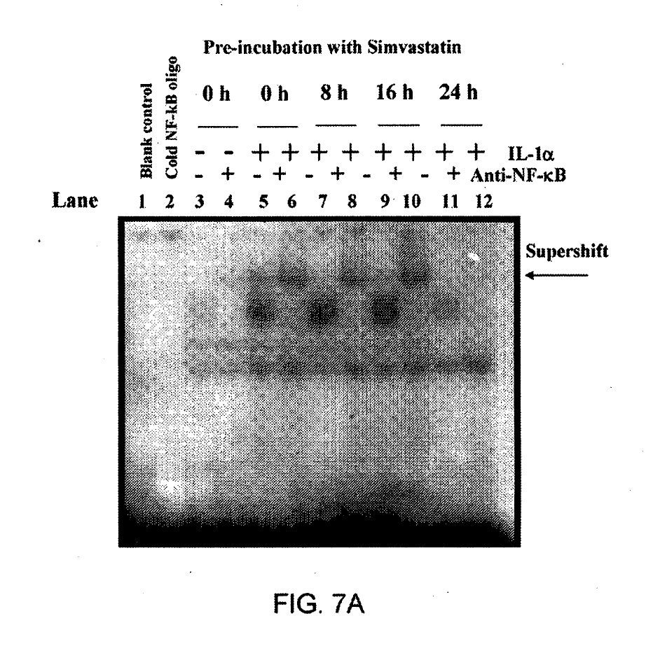

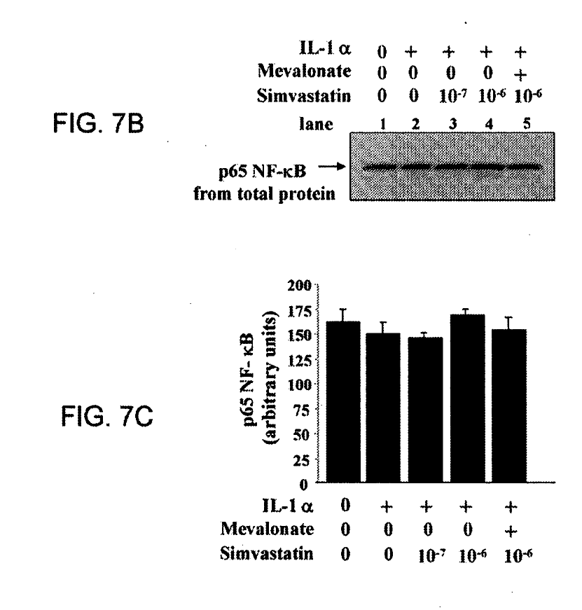

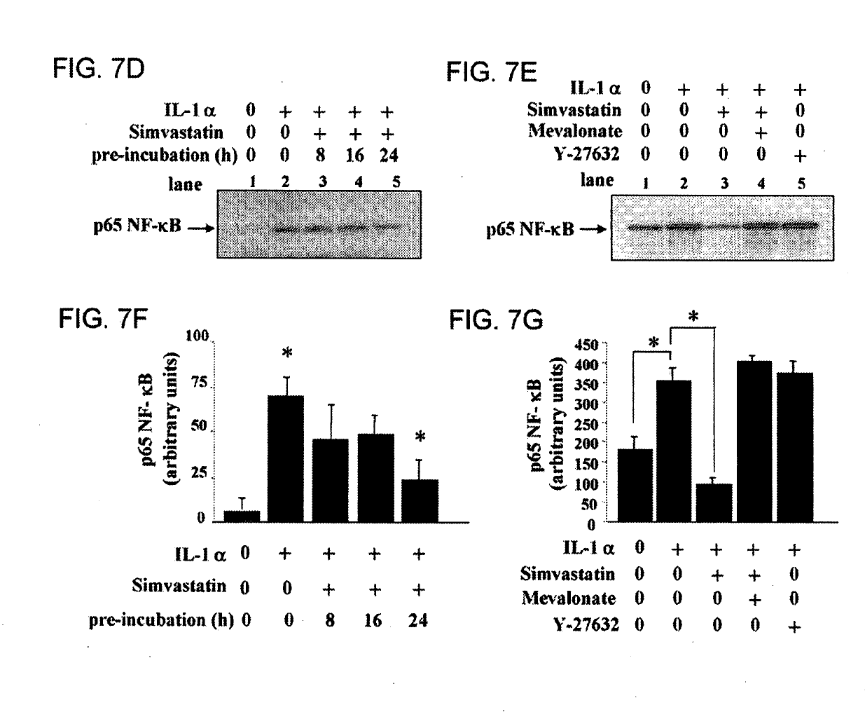

The nuclear transcription factor NF-κB binds to the iNOS gene promoter critical for iNOS gene transcription (25). The effect of simvastatin on IL-1α-induced activation of NF-κB in H9c2 cells was tested by using the gel-shift assay with a ³²P-end-labelled NF-κB oligonucleotide. The nuclear proteins extracted from serum-starved H9c2 cells showed a stronger NF-κB activity after stimulation by IL-1α for 15 min (FIG. 7A). The specificity of the NF-κB DNA-protein complex formation was verified by competition with unlabelled, cold oligonucleotides and by addition of anti-NF-κB antibody which led to the bands for the NF-κB-DNA complexes super-shifted (FIG. 7A). In addition, there was no significant binding with the oligonucleotide probe alone or by omitting protein substrate or using the nuclear protein extract from the unstimulated H9c2 cells. In the subsequent experiments, the cells were treated with 1 μM of simvastatin before IL-1α stimulation. A time-dependent decline in NF-κB-DNA binding was found (FIG. 7A, lane 7 to 10). Simvastatin treatment for 24 hours markedly inhibited IL-1α-induced activation of NF-κB (FIG. 7A, lane 11) (P<0.001 vs IL-1α-stimulated cells). Inclusion of antibody to NF-κB in the binding reactions resulted in a further reduction in the IL-1α-induced mobility of the complex (FIG. 7A, lane 12).

The reduction in the nuclear NF-κB activity did not appear to be due to overall inhibition of expression of this transcription factor by the statin, as immunoblotting analysis of total cellular NF-κB levels showed no significant difference between simvastatin-treated or untreated cells with cytokine stimulation (FIGS. 7B and C). The potential mechanism underlying the simvastatin inhibitory effect on NF-κB activation was delineated by performing immunoblotting analysis on the same nuclear protein extracts from IL-1α-stimulated H9c2 cells with an antibody specific for the p65 subunit of NF-κB. A time-dependent decrease in the p65 NF-κB protein nuclear translocation with pretreatment with simvastatin (FIGS. 7D and F) was observed. The statin-suppressed NF-κB nuclear translocation was clearly related to the HMG CoA reductase activity since addition of L-mevalonate could elevate the nuclear p65/rel protein levels in the statin-treated, cytokine-stimulated cells (FIGS. 7E and G). In addition, the Rho kinase inhibitor Y-27632 showed no major effect on the nuclear NF-κB translocation. Although Y-27632 (10⁻⁴ mol/L) reversed the statin inhibitory effect on iNOS activities, this Rho kinase inhibitor did not influence the nuclear p65 translocation as there was no difference in the nuclear p65 concentration between the cells treated with and without Y-27632 in the presence of IL-1α (FIGS. 7E and G). These observations suggest a discrepancy between L-mevalonate and the Rho inhibitor in regulating the statin modulation of iNOS expression and activation in the cytokine-stimulated cells.

6. Simvastatin Increases the Phosphorylated IκBα in the Cytosol without Subsequent Degradation.

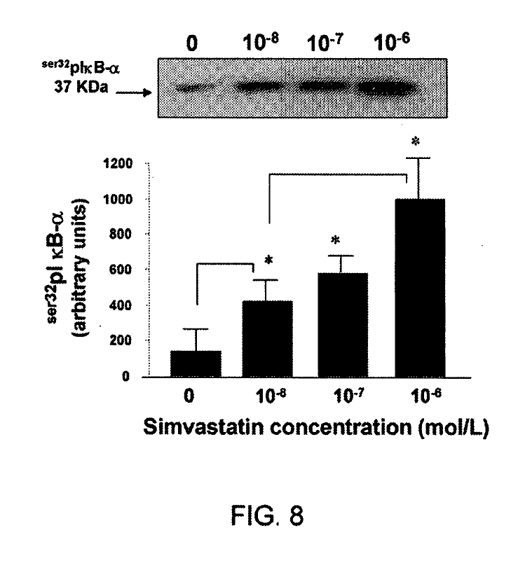

Under the physiological conditions, NF-κB is sequestered as an inactive form in the cytosol through non-covalent interactions with inhibitory proteins, such as IκBα, β and ε. Each IκB isoform contains, in its N-terminal region, a pair of serine residues. In the case of IκBα, these serine residues (amino acids 32 and 36) become phosphorylated by a serine-specific kinase following stimulation. Phosphorylation does not disassociate IκBα from NF-κB but renders IκBα a substrate for ubiquitination and then degradation by the 26S proteasome. In order to provide further insight into the regulatory role of simvastatin, by immunoblotting with a specific antibody to IκBα, the IL-1α-induced degradation of IκBα phosphorylated at Ser⁻³² was examined in the presence or absence of simvastatin. After 15 minutes treatment with 20 ng/mL IL-1α, a decrease in the cellular content of Ser³²-phosphorylated IκBα in H9c2 cells was detected (FIG. 8). However, pretreatment with simvastatin (10⁻⁸ to 10⁻⁶ mol/L) reversed the IL-1α induced decline in the levels of Ser³²-phosphorylated IκBα in a dose-dependent manner (FIG. 8). The highest levels of the phosphorylated IκBα were detected in H9c2 cells exposed to the statin at 10⁻⁷ mol/L for 24 h (FIG. 8). The increased accumulation of phosphorylated IκBα in the simvastatin-treated cells implicates a prolonged lifespan or a lower rate of degradation of phosphorylated IκB protein that inactivates NF-κB.

7. hESC Growth and Differentiation.

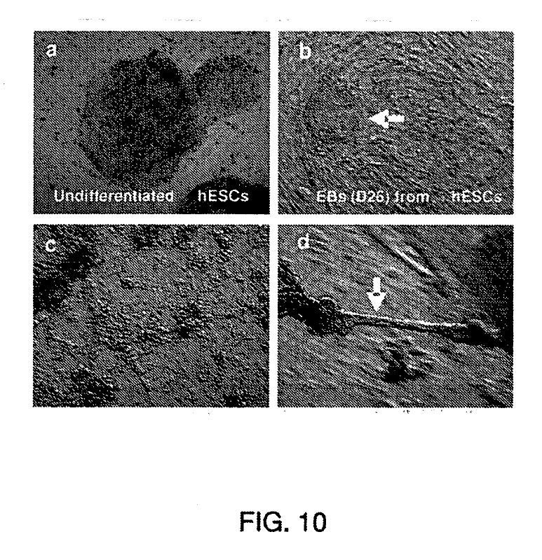

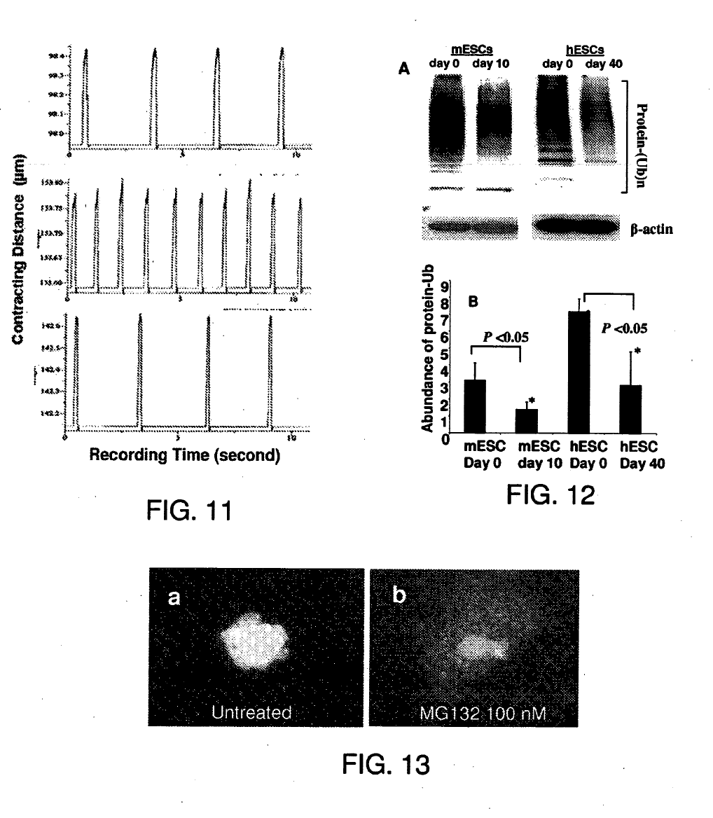

hESC grown in DMEM media with 10% fetal bovin serum and a group of growth factors were induced to differentiate in a hang-drop 3D cell culture system. hESCs from two (H1 or H9) lines were cultured with a mouse fetal fibroblast feeder layer. After forming EBs in the hanging-drop 3D system they were replated in 12-well plates. Cardiovascular phenotypic development was observed in many of the EBs, in which the colonies with beating myocytes developed spontaneously (FIG. 10). Surrounding the beating colonies, the cells developed a blood vessel-like structure (FIG. 10). Using the edge-motion detection technique, a single beating myogenic cell within the developing EBs was recorded. This provides a functional marker showing the differentiational potential in cardiomyogenesis. Expression of β-adrenergic receptors in the developing EBs was evaluated by incubating with the agonist isoproterenol at 10 ng/ml and/or antagonist propanolol at 25 ng/ml. mESC-derived EBs with beating myocytes responded to isoproterenol by marked increased contraction in both frequency and ampltitute (FIG. 11). Addition of propanolol almost abolished the isoproterenol stimulatory effect, suggesting that the stimulatory effect occurred via β-receptor.

8. Protein Ubiquitination in hESC and mESC

Cholesterol is essential for cellular membrane structure, metabolism and function. Cholesterol depletion is lethal to embryogenesis in animals. Cholesterol synthesis is highly regulated by protein phosphorylation and ubiquitination and by sterol-sensitive, SREBP-mediated transcriptional regulation. Several enzymes involved in cholesterol synthesis and metabolism are regulated by the ubiquitin-proteasome system, including HMG-CoA reductase and SREBPs, which are key proteins for cholesterol synthesis. Proteins were extracted from undifferentiated (day 0) murine and human ESCs as well as differentiated (day 10-40). Immunoblotting with anti-ubiquitin antibodies (BD-Bioscience) revealed weaker bands of ubiquitinated proteins in both murine and human ESC at day 0 than those of differentiated murine (day 10) and human (day 40) ESC (FIG. 12). Control loading with anti-b-actin antibody displayed no difference in the β-actin band intensity between the two pairs of cell groups. Thus, protein ubiquitination is more active in undifferentiated ESCs than that in the differentiated ones. The mevalonate pathway leading to cholesterol synthesis also generates non-sterol isoprenoids that have profound biological impacts on various cellular functions, including protein prenylation important for cross-membrane signal transduction and transcriptional regulation.

9. Impact of Protein Ubiquitination on EB Formation

To determine whether protein ubiquitination has any impact on EB formation, hESCs were cultured in hanging-drops with the proteasomal inhibitor MG132 at 100 nm, and then plated in regular culture for 1-2 days. The untreated hESC-derived EB appeared more compact and firmly connected, while the treated EB spread out and surrounded by differentiated cells (FIG. 13). Hence, MG-132 inhibition helped the cell differentiation, suggesting the involvement of ubiquitination in the stem cell differentiation.

10. Conversion of 7-Ketocholesterol to Cholesterol in Human HEK 293 Embryonic Kidney Cells with Overexpression of Apolipoprotein-J (apoJ).

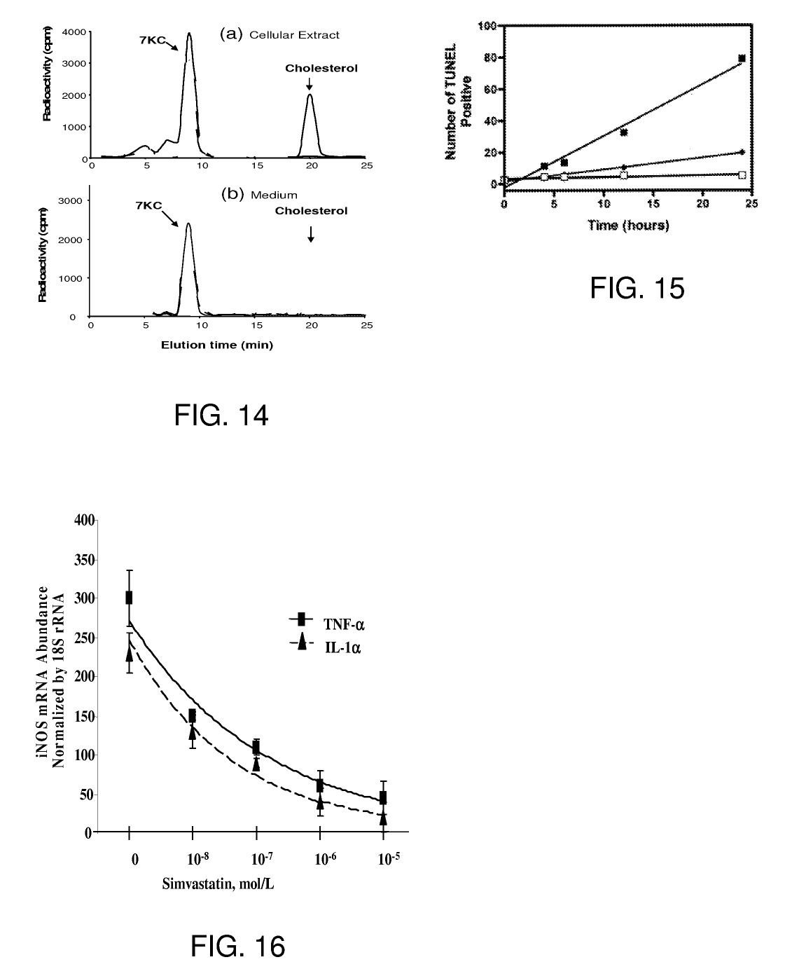

Several embryonic cell lines were recently established with overexpression of apolipoprotein-J, also known as clusterin. Incubation of the cells with radioactive [³H]-7-ketocholesterol (0.5 μci/ml) for 24 hrs led to increased radioactivity in the cells significantly, indicating the uptake of this sterol (FIG. 14). High performance liquid chromatography (HPLC) was performed to separate the cellular and medial lipids, and the radioactivity was measured in the cell lipid extract as well as in culture media. as expected, [³H]-7-ketocholesterol was found in the cells and media at the elution time of about 9 min. Interestingly, free [³H]-cholesterol eluted at nearly 20 min was also detected in the cells but this signal was almost undetectable in the media (FIG. 14). This suggests that the embryonic cells with apoJ overexpression had converted an active metabolism which might convert 7-ketocholesterol into cholesterol.

11. Inhibition of Cholesterol Esterization by ACAT Induces Apoptosis Associated with Cholesterol Transport.

It was tested whether cholesterol accumulation can induce apoptosis in cells treated with the acyl coenzyme-A:cholesterol acyltransferase (ACAT) inhibitor CP-113,818. In situ labeling of DNA fragments were analyzed in murine macrophages treated with the inhibitors using the TUNEL technique. An increase in the number of TUNEL positive cells as a function of the length of incubation of the acLDL-preloaded cells with the ACAT inhibitor CP-113,818 was observed (FIG. 15). However, when treated simultaneously with the hydrophobic amine U18666A, an intracellular cholesterol transport inhibitor and CP-113,818, the number of TUNEL positive cells appearing over time was greatly reduced. U18666A alone had no effect on the number of cells bearing this marker of apoptosis when compared to untreated controls. These TUNEL positive cells showed nuclear morphology typical of apoptosis, including nuclear chromatin condensation and fragmentation. Few TUNEL positive cells were detected in untreated control and in cultures treated with CP-113,818 plus U18666A for 24 h. Thus, inhibition of cholesterol transport with U18666A blocked the apoptotic effect of the ACAT inhibitor.

12. The HMG-CoA Reductase Inhibitor Statin Inhibits iNOS Expression in Embryonic Cardiac Myoblasts Induced by Proinflammatory Cytokines.

In order to determine whether statins affect iNOS expression in embryonic cardiac cells, simvastatin was added into the cell cultures simultaneously with the cytokines. It was observed that in a concentration-dependent fashion, simvastatin markedly diminished expression of iNOS mRNA in H9c2 cells stimulated with the proinflammatory cytokines (FIG. 16). Under the same concentrations, both IL-1α and TNF-α stimulated cells showed a similar response to simvastatin in terms of iNOS mRNA expression. In the presence of simvastatin, IL-1α treated cells showed a dose-dependent decline in iNOS mRNA to the same or similar degrees as that in TNF-α treated cells. Thus, simvastatin reduced steady-state levels of iNOS mRNA in H9c2 cardiac myoblasts stimulated with the proinflammatory cytokines.

13. The Isoprenoid Intermediate GGPP Blocks Simvastatin Inhibitory Effect on iNOS Expression.

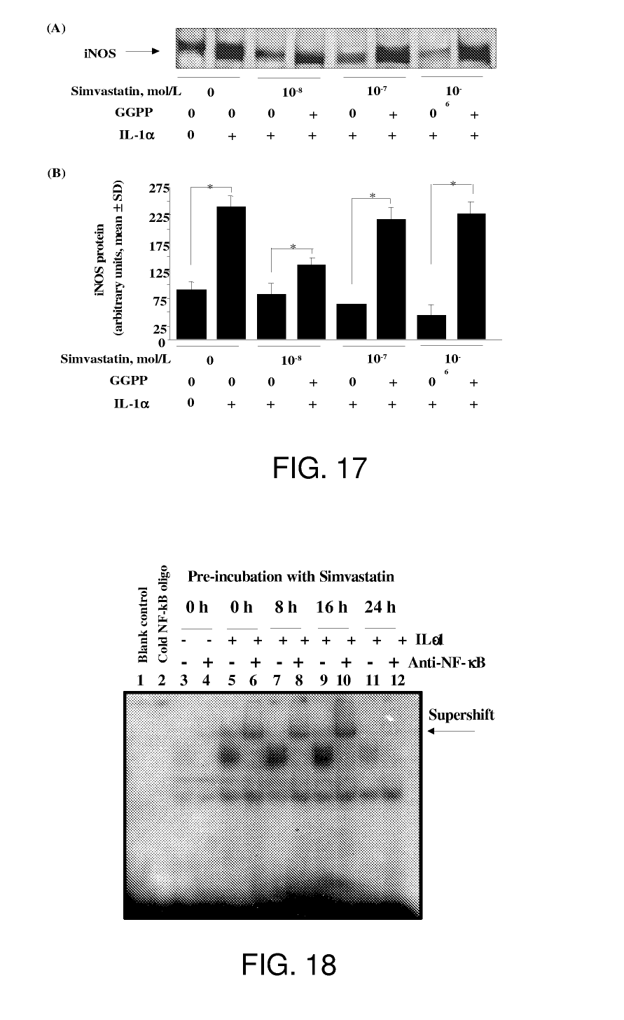

In addition to L-mevalonate, several downstream intermediates or by-products from the cholesterol biosynthetic pathway may contribute to the inhibitory effect of simvastatin on expression of iNOS protein and activities. H9c2 cells were pretreated with simvastatin (10⁻⁸ to 10⁻⁷ mol/L) up to 24 h, and then stimulated the cells with IL-1α (20 ng/mL) in the presence or absence of the isoprenoid intermediates, such as GGPP and FPP. Immunoblotting showed that addition of GGPP (10⁻⁷ to 10⁻⁵ mol/L) reversed the simvastatin-mediated suppression of the cytokine-induced expression of iNOS protein (FIGS. 17A-B) at almost the same levels to those in the L-mevalonate treated cells. In contrast, however, treatment with FPP at the same concentrations as GGPP did not alter nitrite accumulation in the cells pretreated with simvastatin and then IL-1α (not shown). These results suggested that GGPP but not FPP appeared to directly reverse the statin-induced inhibition of iNOS expression in the cardiac myoblasts stimulated with IL-1α.

14. Simvastatin Inactivates NF-κB in Cardiac Myoblasts Stimulated with Cytokines.

The nuclear transcription factor NF-κB binds to the iNOS gene promoter critical for iNOS gene transcription. The effect of simvastatin on IL-1α-induced activation of NF-κB in H9c2 cells was examined by using the gel-shift assay with a ³²P-end-labelled NF-κB oligonucleotide. The nuclear proteins extracted from serum-starved H9c2 cells showed a stronger NF-κB activity after stimulation by IL-1α for 15 min (FIG. 18.). The specificity of the NF-κB DNA-protein complex formation was verified by competition with unlabelled, cold oligonucleotides and by addition of anti-NF-κB antibody which super-shifted the bands of NF-κB-DNA complexes (FIG. 18.). In addition, there was no significant binding with the oligonucleotide probe alone or by omitting protein substrate or using the nuclear protein extract from the unstimulated H9c2 cells. In the subsequent experiments, the cells were treated with 1 μM of simvastatin before IL-1α stimulation. A time-dependent decline in NF-κB-DNA binding was found (FIG. 18, lane 7 to 10). Simvastatin treatment for 24 hours markedly inhibited IL-1α-induced activation of NF-κB (FIG. 18, lane 11) (P<0.001 vs IL-1α-stimulated cells). Inclusion of antibody directed against NF-κB in the binding reactions resulted in a further reduction in the IL-1α-induced mobility of the complex (FIG. 18, lane 12). The reduction in the nuclear NF-κB activity did not appear to be due to overall inhibition of expression of this transcription factor by the statin, as immunoblotting analysis of total cellular NF-κB levels showed no significant difference between simvastatin-treated or untreated cells with cytokine stimulation.

15. Simvastatin Increases the Phosphorylated IκBα in the Cytosol.

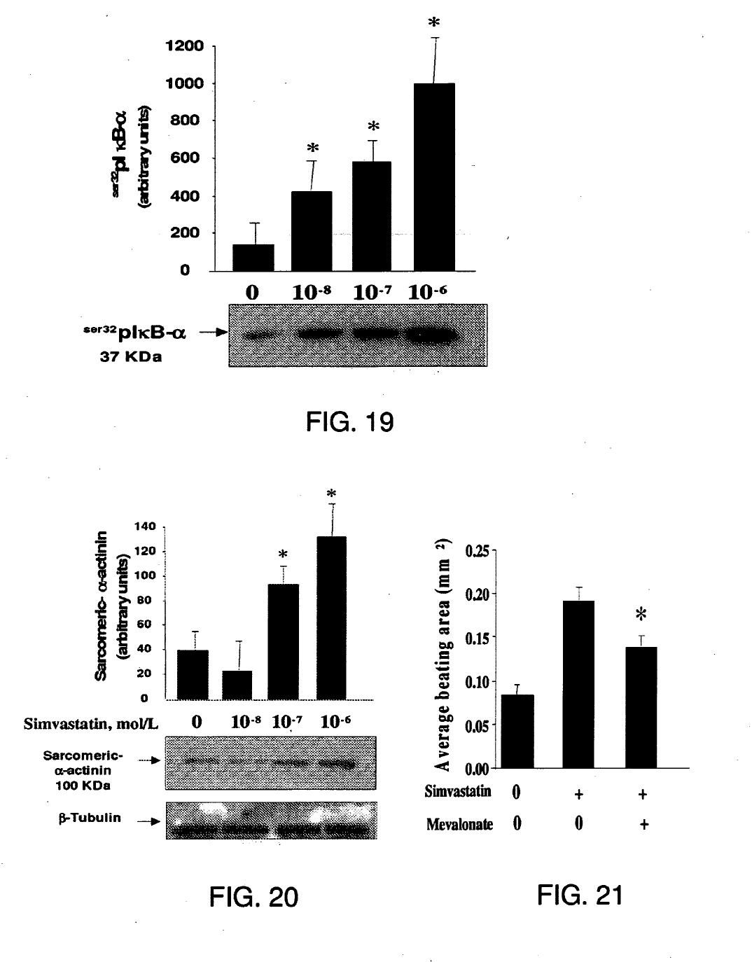

Under the physiological conditions, NF-κB is sequestered as an inactive form in the cytosol through non-covalent interactions with inhibitory proteins, such as IκBα, β and ε. Each IκB isoform contains, in its N-terminal region, a pair of serine residues. In the case of IκBα, these serine residues (amino acids 32 and 36) become phosphorylated by a serine-specific kinase following stimulation. Phosphorylation does not disassociate IκBα from NF-κB but renders IκBα a substrate for ubiquitination and then degradation by the 26S proteasome. In order to provide further insight into the regulatory role of simvastatin, by immunoblotting with a specific antibody to IκB, the IL-1α-induced degradation of IκBα phosphorylated at Ser³² in the presence or absence of simvastatin was observed. After 15 minutes treatment with 20 ng/mL IL-1α, a decrease in the cellular content of Ser³²-phosphorylated IκBα in H9c2 cells was observed. However, in the cytokine-stimulated cells, pretreatment of with simvastatin (10⁻⁸ to 10⁻⁶ mol/L) increased the levels of Ser³²-phosphorylated IκBα in a dose-dependent manner (FIG. 19). The highest levels of the phosphorylated IκBα were detected in H9c2 cells exposed to the statin at 10⁻⁷ mol/L for 24 h (FIG. 19). The increased accumulation of phosphorylated IκBα in the simvastatin-treated cells implicates a prolonged lifespan or a lower rate of degradation of phosphorylated IκB protein that inactivates NF-κB.

16. Simvastatin Promotes Cardiomyogenic Differentiation of Embryonic Stem Cells

It was tested whether statin treatment has any impact on myogenesis in embryonic stem cells. Western blot was conducted in simvastatin-treated and untreated murine embryonic stem cells with monoclonal antibodies against cardiac sarcomeric α-actinin. Stem cells were cultured in a hanging drop system for 4 days and then transferred to petric dishes for further development. A dose-dependent increase in cardiac myogenesis was found, evidenced by increased expression of cardiac sarcomeric α-actinin (FIG. 20) and appearance of contractile myocytes (FIG. 21). The induction of beating myocytes in the statin-treated ESCs could be partially blocked by L-mevalonate.

17. Potential Applications for Non-Sterol Isoprenoid Intermediates

Cholesterol synthesis generates non-sterol isoprenoid intermediates as by-products, which can promote protein prenylation as well as signal transduction potentially important for stem cell growth, survival and differentiation as well as atherosclerosis and inflammation (62-64,83,84). Abnormal synthesis and metabolism of cholesterol may cause certain severe pathological conditions. For instance, hypercholesterolemia is a causitive risk factor for atherosclerosis (85), a chronic arterial disease with life-threatening complications, namely myocardial and cerebral infarctions, which is the leading cause of death in the United States, while hypocholesterolemia characterizes the Smith-Lemli-Opitz syndrome (SLOS) (75,76,85), a recessive autosomal genetic disease characterized by a deficit in cholesterol production with a series of malformations (microcephaly, corpus callosum agenesis, holoprosencephaly, and mental retardation), male pseudohermaphroditism, finger anomalies, and failure to thrive.

Little is known, however, about the potential role of cholesterol biosynthesis and metabolism in regulation of hESC function, and the hESC pluripotency in growth, survival and differentiation. It is proposed that hESCs undergo active cholesterol synthesis and metabolism, and that regulation of the production of cholesterol and its derivatives plays a critical role for the maintenance of hESC pluripotency in proliferation, survival and differentiation under both physiological or pathophysiological conditions. At least some of the existing cholesterol-lowering drug statins may have potentially beneficial effects on hESCs, apart from their customery use. Millions of patients are taking one of the statin drugs now for prevention and treatment of atherosclerosis, but the biosafety of this HMG-CoA reductase inhibitor is still a concern among health providers and patients. In this regard, hESCs provide a highly valuable model for testing the effect of statins on human embryonic development. Because the isoprenoids prenylate a number of membrane-bound or receptor-associated proteins important for cell signal transduction, it is now proposed for the first time that one or more non-sterol isoprenoid intermediate of cholesterol synthesis or mevanolate/isoprenoid pathway inhibitors will be potentially useful as a therapeutic drug, and can be used in combination with stem cell therapy. The intermediate compounds geranylgeranyl pyrophosphate (PPGG) and geranyl pyrophosphate (PGG), for example, are expected to modulate apoptosis in stem cells, myoblasts and other cells by isoprenoid-mediated signaling transduction. Potential applications include protective effects for stem cells, and treatment of a variety of degenerative diseases, wound healing and cancer treatment. It is expected that further analysis of prenylated membrane proteins and cell signaling in hESCs will demonstrate the feasibility of this approach to development of this type of new, non-sterol therapeutic compounds.

18. Effects of Simvastatin on Cardiomyocyte Differentiation

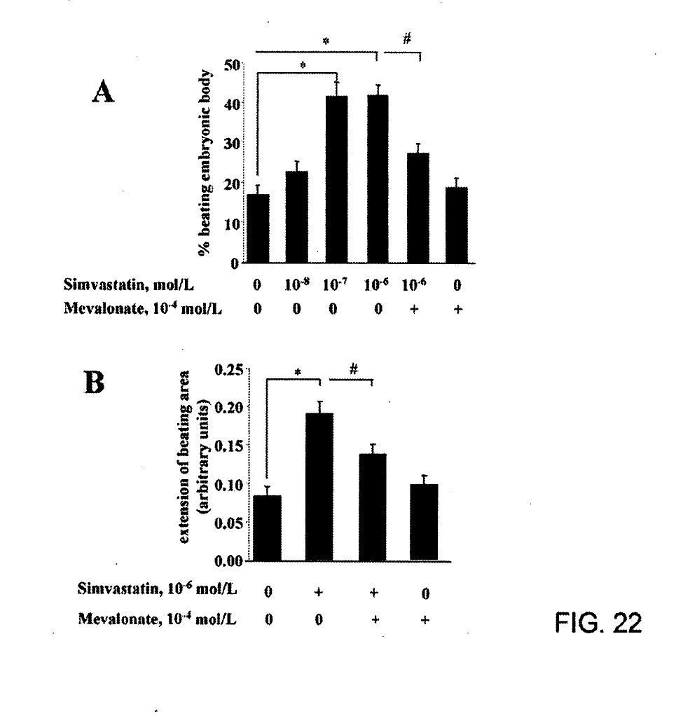

Spontaneous cardiac differentiation of mouse embryonic stem cells (mESCs) was assessed in vitro as the presence of rhythmically beating embryonic blastocyst (EB) outgrowth. To determine the effects of cholesterol depletion on differentiation by blocking the HMG-CoA reductase activity with statin, some cultures were supplemented with simvastatin (10⁻⁸-10⁻⁶ mol/L) after plating the EBs from the hanging drops (day zero). Thereafter, EB outgrowths were counted periodically under the inverted microscope equipped with a digital video camera, to determine whether or not they contained beating foci at different time points (days 3, 5, 7, 10, 12, and 14). At approximately day 7, contracting areas began to appear. There was no major difference in the size and beating foci between the simvastatin-treated and untreated EBs, indicating that the statin treatment did not prevent EB formation and early myogenesis. At day 12, simvastatin exposure increased the percentage of beating EB outgrowths (42±0.1%) compared to that of the untreated control EBs (18±0.1%) (FIG. 22A). Concomitant with the increased number of beating EBs, the size of the beating area for simvastatin-treated EBs increased 2-fold (evaluated at day 12) compared to that of untreated control EBs, a significant augmentation (p<0.05) (FIG. 22B).

To determine whether the statin induction of contracting EBs occurred by blocking the HMG-CoA reductase activity or inhibiting L-mevalonate synthesis (a key intermediate of cholesterol synthesis from acetyl-CoA), exogenous L-mevalonate was added into the cultures of EBs in the presence or absence of 10⁻⁶ mol/L simvastatin. It was observed that adding excess L-mevalonate significantly decreased the incidence of beating foci in the EBs exposed to simvastatin—without any cytotoxicity (FIG. 22A). However, in the absence of simvastatin, L-mevalonate had no or little effect on contracting EBs (FIG. 22A), suggesting that L-mevalonate selectively blocked the simvastatin stimulatory effect on cardiac differentiation. All analyzed EB outgrowths showed a similar developmental pattern with contracting cells first appearing at the periphery of EB outgrowths.

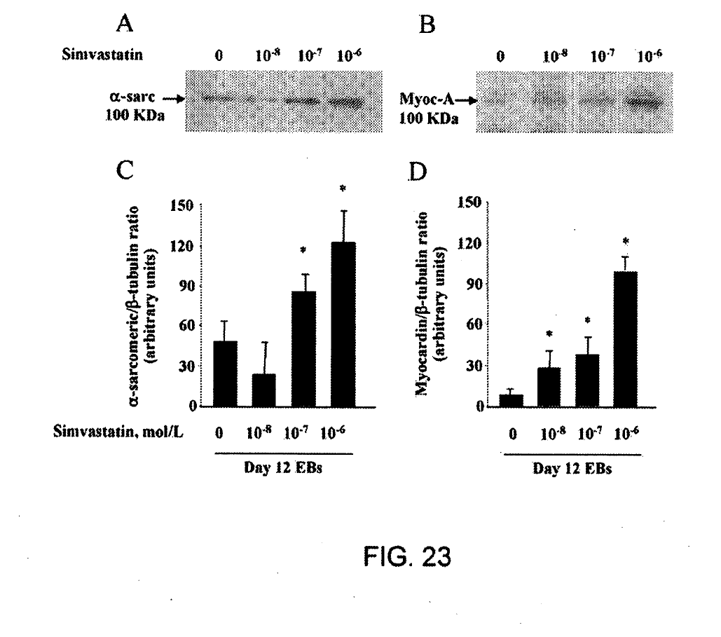

To establish the phenotypic characteristics of EB outgrowths, expression of cardiac-specific proteins was assessed by immunoblotting with antibodies against sarcomeric α-actinin and myocardin A, a key regulator of cardiac myogenesis (57). By day 12, in a dose-dependent fashion, simvastatin treatment increased expression of both sarcomeric α-actinin (FIGS. 23A and C) and myocardin A (FIGS. 23B and D), while the statin had little impact (not shown) on expression of Tie-2, an endothelian cell-specific protein potentially important for vascular tissue formation (58).

Under the same cell culture conditions specific for cardiomyocyte differentiation, untreated EBs showed modest expression of sarcomeric α-actinin, and myocardin A (FIG. 23A, 23B). Thus, treatment with simvastatin appeared to enhance expression of cardiac-specific proteins in a concentration-dependent manner. The range of simvastatin concentrations leading to induction of cardiac differentiation is comparable to the drug plasma levels in patients treated with the statin (20).

19. Effects of Simvastatin on Apoptosis of Embryonic Myoblasts Induced by IL-1

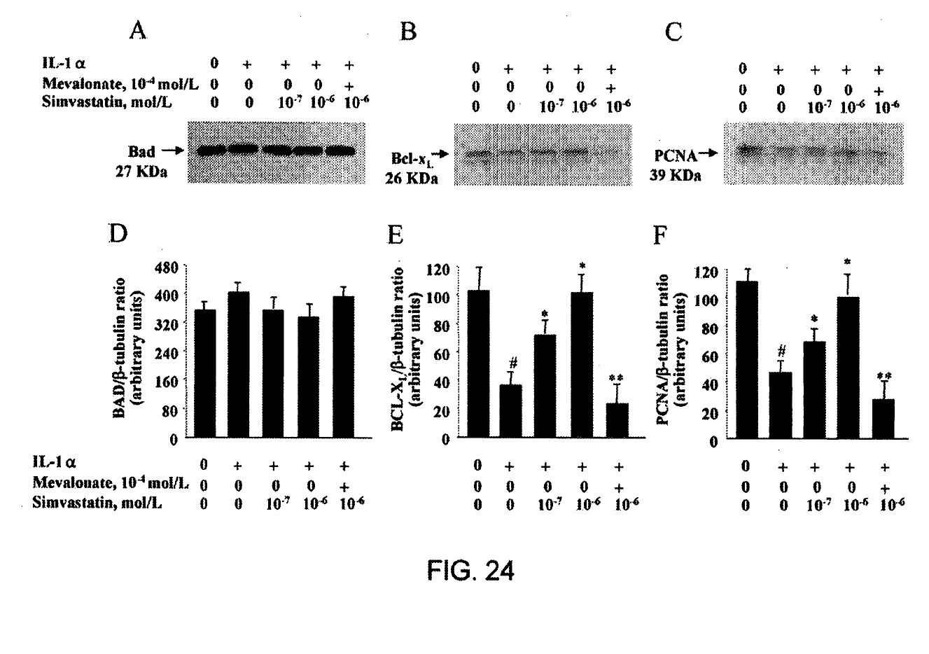

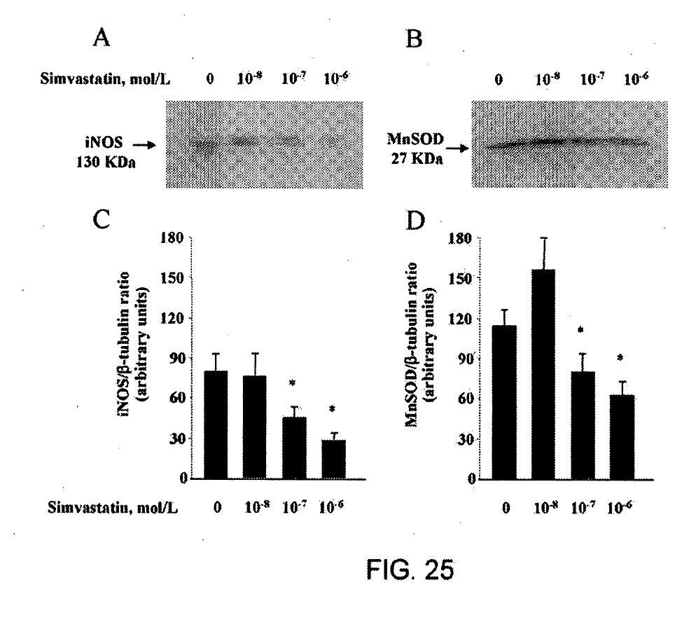

It was previously demonstrated that simvastatin treatment can attenuate iNOS expression and NO synthesis in cytokine-stimulated embryonic cardiac myoblasts (45). Because the high output of NO production is pro-apoptotic, it was hypothesized whether simvastatin can increase the resistance of myogenic cells against apoptosis induced by IL-1, a proinflammatory cytokine. The committed H9C2 embryonic myoblasts were pretreated with simvastatin (10⁻⁷-10⁻⁶ mol/L), and then IL-1 (20 ng/mL) was added into the cultures to trigger apoptosis. The proapoptotic protein Bad and anti-apoptotic protein Bcl-x_{L} (59,60) wwere analyzed by immunoblotting. In the cells without statin treatment, IL-1 reduced expression of the anti-apoptotic protein Bcl-x_{L} but had no significant effects on Bad expression (FIGS. 24A and 24B). Adding simvastatin (10⁻⁷-10⁻⁶ mol/L) significantly diminished the IL-1 inhibitory effects. The simvastatin effect appeared to be mediated by the mevalonate pathway because adding L-mevalonate reversed the statin effect (FIG. 24B). The proliferating cell nuclear antigen was also down-regulated by IL-1 treatment, which could be partially blocked by adding simvastatin under a mechanism controlled by mevalonate (FIG. 24C). Treatment with simvastatin significantly diminished expression of iNOS (FIG. 25A) and MnSOD (FIG. 25B).

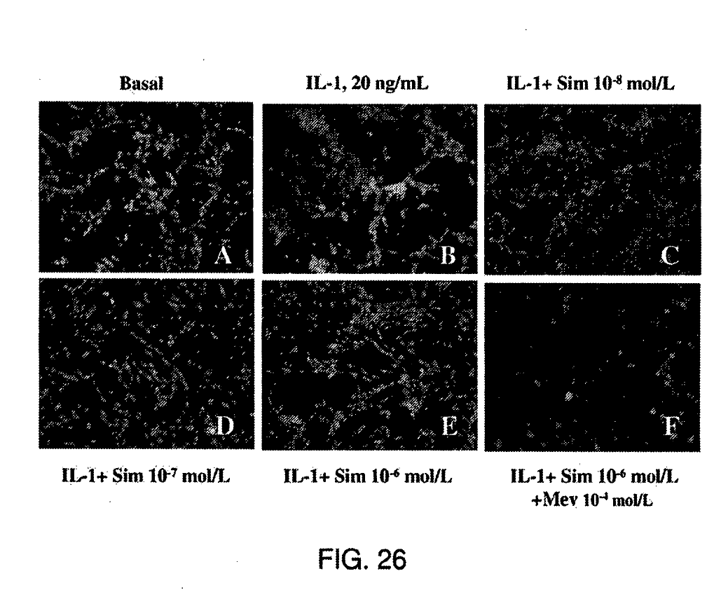

Furthermore, it was examined whether simvastatin exerts protective effects against apoptosis induced with IL-1 by staining with the fluorochromes acridine orange and ethidium bromide in H9c2 embryonic myoblasts. In untreated cells, low rates (less than 5%) of apoptosis occurred spontaneously (FIG. 26, Panel A). However, exposure to the cytokine for a prolonged period of time led to increased apoptosis (FIG. 5, Panel B). Incubation with IL-1 (20 ng/mL) for 48 to 72 hours induced significant elevation of cell death by 2- to 3-fold (p<0.05). Interestingly, in agreement with a previous finding (45), simvastatin significantly increased cell viability and reduced numbers of apoptotic cells in the cultures with IL-1 (FIG. 26, Panels C-E). The simvastatin cytoprotective effect was diminished by L-mevalonate (FIG. 26, Panel F).

Thus, simvastatin may not only induce myogenic differentiation but also protect differentiated cardiac cells and premature embryonic cardiomyoblasts against cytokine-induced apoptosis. Simvastatin is a representative statin with biological activities shared by other statins, such as atorvastatin, cerivastatin, fluvastatin, lovastatin, mevastatin, pitavastatin, rosuvastatin, and ezetimibe, which are expected to provide effects similar to those described above.

Discussion

The embryonic H9c2 cardiac myoblast model has been used widely to study cardiac stem cell development and differentiation. Highly resembling premature cardiac myogenic cells, H9c2 cells show well-characterized cardiac properties in electrophysiology as well as in cellular receptor and signal transduction (26,27). It is notable that the embryonic H9c2 cardiac myoblasts can constitutively express iNOS as well as eNOS at low levels under the normal cell culture. The constitutively expressed, moderate NOS activities may reflect the role of NO in intracellular signalling of undifferentiated, premature myoblasts. Bloch et al. (28,29) have reported that both iNOS and eNOS exist in E9.5 rat and murine embryos, correlated with high expression of soluble guanylylcyclase as well as a high cyclic GMP content. The NO production mediated by the NOS isoforms present constitutively in the cardiac myogenic cells contributes to cardiomyogenesis, since continuous incubation of EBs with the NOS inhibitors results in a pronounced differentiation arrest in the premature cardiomyocytes, and coapplication of NO-donors reverses the inhibitory effect.

However, the high-output, persistent NO production via overexpression of iNOS induced by proinflammatory cytokines may have deteriorating effects on cardiac cells (8,13). In this study, it was shown that the H9c2 premature cardiac cell line is sensitive to stimulation of proinflammatory cytokines such as IL-1α and TNFα. The high levels of iNOS expression and NO production in this premature, delicate myocyte progenitor suggests a vulnerability of cardiac stem cells to the proinflammatory environment of the hearts with acute infarction or ischemic injury. Because the high output of NO production causes cardiac cell dysfunction and even apoptotic cell death, inhibition of iNOS expression may have benefit impacts on the stem cell survival and differentiation. Increased resistance to proinflammatory or pro-apoptotic insults may represent a key factor that leads to a successful cardiac stem cell therapy. Current data from the in vitro studies provide clear evidence that the cholesterol-lowering drugs, statins, can regulate iNOS expression. The statin regulatory effect on iNOS expression appears through specific inhibition of HMG CoA reductase, reversibly blocked by excess amounts of L-mevalonate. The statin effect seems however independent of the statin-mediated reduction in cholesterol synthesis because addition of exogenous cholesterol does not prevent the statin effect. The concentrations for simvastatin achieving its inhibitory effect are as low as 10⁻⁸ mol/L. The dosage of 10⁻⁸-10⁻⁶ mol/L is close to the range of the expected plasma levels of simvastatin in clinical application (20). This suggests that statins, at the current therapeutic dosages, may block iNOS expression during inflammation.

In this study, the mechanism by which statins regulate iNOS expression was demonstrated, showing that simvastatin acts multilaterally at different phases of iNOS expression, and on the levels of mRNA, protein and enzymatic activities. Recently, the isoprenoid intermediates, including GGPP and FPP, have been implicated in contributing to the statin regulatory effects on expression of NOS expression. This is largely due to the fact that addition of GGPP significantly can diminish the statin-inhibitory effect. It has been previously demonstrated that, in addition to reducing hepatic cholesterol biosynthesis by inhibiting HMG CoA reductase, statins may display biological activities associated with depletion of L-mevalonate as well as biologically active isoprenoid intermediates. Some of the isoprenoids (e.g., GGPP) play an important role in the covalent attachment to cell membranes, subcellular localization, and intracellular trafficking of membrane-associated proteins, including regulating endothelial cell function (30). In this study it was observed that the addition of GGPP, but not FPP, prevented the inhibitory effect of simvastatin on cytokine-induced iNOS expression and activity, indicating that the inhibition of post-translational geranylgeranylation, but not of farnesylation, can be related to the inhibitory effect of simvastatin on cytokine-induced iNOS expression in the premature myoblasts.

The Rho proteins (23) are a group of small GTP-binding molecules involved in the control of NOS protein expression and turnover in cardiovascular cells (11,31,32). The members of the Rho family including RhoA, RhoB, Rac and Cdc42 proteins are normally geranylgeranylated, whereas Ras proteins are predominantly farnesylated. Current data from the studies with the Rho kinase inhibitor point to the involvement of Rho proteins in simvastatin-associated iNOS inhibition in premature cardiac myoblasts. Rho kinase activation has been implicated to reduce iNOS activities. In this study, it was found that inhibition of Rho kinase with Y-27632 enhances NO production in cytokine-stimulated H9c2 cells. Furthermore, with enzymatic assays, it was demonstrated that the statin treatment can reduce the Rho kinase activity as well. This finding is consistent with previous reports that lipid-soluble statins (e.g., lovastatin, simvastatin, and atorvastatin) inhibit the Rho kinase activity (33). However, from the data generated in this study, the simvastatin treatment did not appear to cause superinduction but suppression of iNOS expression by IL-1 in H9c2 cells, unlike the previous study conducted in vascular smooth muscle cells (33). The different statin effects between the embryonic cardiac myoblasts and mature smooth muscle cells may reflect the fact that different signal transduction pathways may operate in different types of cells.

Pretreatment with lovastatin, an inhibitor of protein prenylation, resulted in superinduction of iNOS. This superinduction can be reversed by geranylgeraniol, but not by farnesol, suggesting that inhibition of geranylgeranylation, not farnesylation, is responsible for enhanced iNOS expression. The results demonstrate that a farnesylated protein(s) mediates IL-1beta induction of iNOS, whereas a geranylgeranylated protein(s) represses this induction. Also, by modulating Ras farnesyl protein transferase, lovastatin blocks LPS-induced iNOS expression in rat primary astrocytes in a manner reversible by L-mevalonate and farnesylpyrophosphate (14). However, on the contrary, treatment with cerivastatin and fluvastatin was recently reported to enhance cytokine induction of NO synthesis in rat vascular smooth muscle cells (16). An increase in IL-1β-induced nitrite production and apoptosis in adult cardiac myocytes may occur after treatment with very high concentrations (10⁻⁵ and 10⁻⁴ mol/L) of fluvastatin through inhibition of Rho-associated kinase (34,35). In addition, there have been reports showing that statin treatment may induce apoptosis of vascular cells (35-37). However, in the present system, no appreciable cell death via apoptosis was observed in the statin-treated cardiac myoblasts, regardless of the presence or absence of the proinflammatory cytokines. Recent studies (38,39) have also demonstrated that statin treatment can increase numbers of circulating endothelial cell progenitors, suggesting that statins are cytoprotective rather than cytotoxic to the undifferentiated, embryonic stem cells.

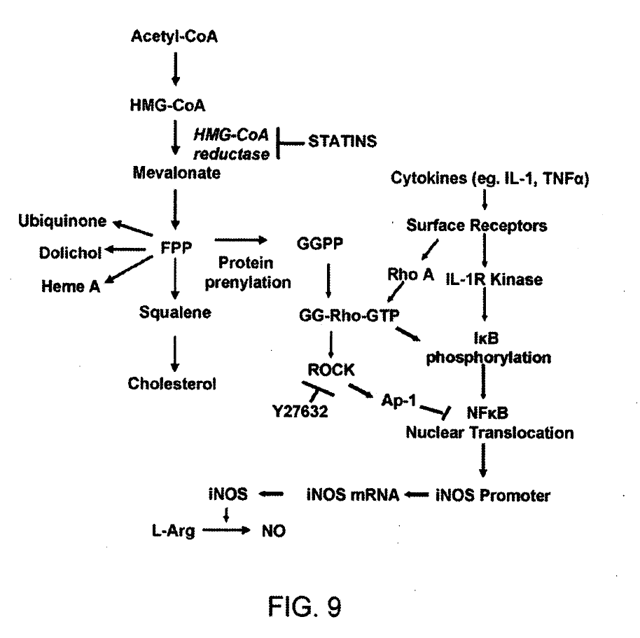

The cytokine induction of iNOS expression involves multiple transcription factors, in particular the nuclear factor NF-κB. Upon activation by cytokines (e.g., IL-1 and TNF-α, NF-κB translocates from the cytosolic compartment to the nucleus, where it binds to the iNOS gene promotor, and ultimately triggers iNOS gene transcription. This process is mediated by kinase-mediated protein phosphorylation and requires disassociation between NF-κB and its native inhibitor IκB. One of the statins, mevastatin, has been reported to inactivate NF-κB and reduce NF-κB-dependent expression of the endothelial adhesion proteins, VCAM and E-selectin (40). Consistently, the data from the current study clearly shows that simvastatin treatment can reduce NF-κB nuclear translocation and elevate the cytosolic content of phosphorylated IκBα. Rho associated kinases (ROCK) have been reported to mediate the regulatory effect of statins on iNOS expression in airway epithelial cells (41). ROCK may control the iNOS gene promoter activities via NF-κB, however, the ROCK inhibitor Y-27632 shows different effects from those of statins in terms of iNOS gene activation. Thus, it is likely that statins may regulate cytokine-induced iNOS expression through different signal pathways which involve both Y-27632-sensitive and insensitive signalling as well as the NF-κ-B and IκBα interaction (FIG. 9).

At the present time, there is still a dispute in this field as to whether NO protects or damages the myocardium during inflammation or whether statin suppression of iNOS expression is beneficial or harmful to the heart (2). It has been recently established that simvastatin reduces reperfusion injury in the isolated-perfused working rat heart by preventing eNOS from inactivation and by suppressing ischemia-related iNOS induction, which correlates with a reduction in cardiomyocyte apoptosis (17). The current finding that simvastatin decreases cytokine-induced iNOS expression in cultured premature cardiac myoblasts illustrates a causal relationship between increased NO and depressed cardiac contractility in heart failure. Further clarification of the mechanism underlying the statin effect on NO production and survival of cardiac stem cells may provide valuable information that will help in designing therapies for patients with myocardial infarction or ischemic heart failure.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Accordingly, the scope of protection is not limited by the description set out above, but is only limited by the claims, that scope including all equivalents of the subject matter of the claims. The disclosures of all patents, patent applications and publications cited herein are hereby incorporated herein by reference to the extent that they describe materials, methods or other details supplementary to those set forth herein.

### REFERENCES

The following literature is cited by number in the foregoing text:
1. Geng, Y. J. (2003) Ann N Y Acad Sci 1010, 687-697
2. Massion, P. B., Feron, O., Dessy, C., and Balligand, J. L. (2003) Circ Res 93, 388-398
3. Kim, S. J., Kim, Y. K., Takagi, G., Huang, C. H., Geng, Y. J., and Vatner, S. F. (2002) Am J Physiol Heart Circ Physiol 282, H423-428
4. Geng, Y., Hansson, G. K., and Holme, E. (1992) Circ Res 71, 1268-1276
5. Geng, Y. J., Almqvist, M., and Hansson, G. K. (1994) Biochim Biophys Acta 1218, 421-424
6. Geng, Y. J., Hellstrand, K., Wennmalm, A., and Hansson, G. K. (1996) Cancer Res 56, 866-874
7. Birks, E. J., Yacoub, M. H., Burton, P. S., Owen, V., Pomerance, A., O'Halloran, A., Banner, N. R., Khaghani, A., and Latif, N. (2000) Transplantation 70, 1498-1506
8. Birks, E. J., and Yacoub, M. H. (1997) Coron Artery Dis 8, 389-402
9. Gotto, A. M., Jr. (2001) Am J Cardiol 87, 13B-18B
10. Gotto, A. M., Jr. (2001) Am J Cardiol 88, 36F-40F
11. Laufs, U., Kilter, H., Konkol, C., Wassmann, S., Bohm, M., and Nickenig, G. (2002) Cardiovasc Res 53, 911-920
12. Wassmann, S., Laufs, U., Baumer, A. T., Muller, K., Ahlbory, K., Linz, W., Itter, G., Rosen, R., Bohm, M., and Nickenig, G. (2001) Hypertension 37, 1450-1457
13. von Haehling, S., Anker, S. D., and Bassenge, E. (2003) Heart Fail Rev 8, 99-106
14. Pahan, K., Sheikh, F. G., Namboodiri, A. M., and Singh, I. (1997) J Clin Invest 100, 2671-2679
15. Pahan, K., Liu, X., McKinney, M. J., Wood, C., Sheikh, F. G., and Raymond, J. R. (2000) J Neurochem 74, 2288-2295
16. Hattori, Y., Nakanishi, N., and Kasai, K. (2002) Cardiovasc Res 54, 649-658
17. Di Napoli, P., Antonio Taccardi, A., Grilli, A., Spina, R., Felaco, M., Barsotti, A., and De Caterina, R. (2001) Cardiovasc Res 51, 283-293
18. Geng, Y. J., Petersson, A. S., Wennmalm, A., and Hansson, G. K. (1994) Exp Cell Res 214, 418-
19. Geng, Y. J., Wu, Q., Muszynski, M., Hansson, G. K., and Libby, P. (1996) Arterioscler Thromb Vasc Biol 16, 19-27
20. Desager, J. P., and Horsmans, Y. (1996) Clin Pharmacokinet 31, 348-371
21. Takemoto, M., and Liao, J. K. (2001) Arterioscler Thromb Vasc Biol 21, 1712-1719
22. Mackay, D. J., Esch, F., Furthmayr, H., and Hall, A. (1997) J Cell Biol 138, 927-938
23. Mackay, D. J., and Hall, A. (1998) J Biol Chem 273, 20685-20688
24. Ishizaki, T., Uehata, M., Tamechika, I., Keel, J., Nonomura, K., Maekawa, M., and Narumiya, S. (2000) Mol Pharmacol 57, 976-983
25. Xie, Q. W., Kashiwabara, Y., and Nathan, C. (1994) J Biol Chem 269, 4705-4708
26. Hescheler, J., Meyer, R., Plant, S., Krautwurst, D., Rosenthal, W., and Schultz, G. (1991) Circ Res 69, 1476-1486
27. Hescheler, J., Fleischmann, B. K., Wartenberg, M., Bloch, W., Kolossov, E., Ji, G., Addicks, K., and Sauer, H. (1999) Cells Tissues Organs 165, 153-164
28. Bloch, W., Fleischmann, B. K., Lorke, D. E., Andressen, C., Hops, B., Hescheler, J., and Addicks, K. (1999) Cardiovasc Res 43, 675-684
29. Bloch, W., Addicks, K., Hescheler, J., and Fleischmann, B. K. (2001) Microsc Res Tech 55, 259-269
30. Vincent, L., Chen, W., Hong, L., Mirshahi, F., Mishal, Z., Mirshahi-Khorassani, T., Vannier, J. P., Soria, J., and Soria, C. (2001) FEBS Lett 495, 159-166
31. Laufs, U., Endres, M., Custodis, F., Gertz, K., Nickenig, G., Liao, J. K., and Bohm, M. (2000) Circulation 102, 3104-3110
32. Laufs, U., Gertz, K., Huang, P., Nickenig, G., Bohm, M., Dirnagl, U., and Endres, M. (2000) Stroke 31, 2442-2449
33. Finder, J. D., Litz, J. L., Blaskovich, M. A., McGuire, T. F., Qian, Y., Hamilton, A. D., Davies, P., and Sebti, S. M. (1997) J Biol Chem 272, 13484-13488
34. Ikeda, Y., Young, L. H., and Lefer, A. M. (2003) J Cardiovasc Pharmacol 41, 649-656
35. Ogata, Y., Takahashi, M., Takeuchi, K., Ueno, S., Mano, H., Ookawara, S., Kobayashi, E., Ikeda, U., and Shimada, K. (2002) J Cardiovasc Pharmacol 40, 907-915
36. Guijarro, C., Blanco-Colio, L. M., Ortego, M., Alonso, C., Ortiz, A., Plaza, J. J., Diaz, C., Hernandez, G., and Egido, J. (1998) Circ Res 83, 490-500
37. Kaneta, S., Satoh, K., Kano, S., Kanda, M., and Ichihara, K. (2003) Atherosclerosis 170, 237-243
38. Llevadot, J., Murasawa, S., Kureishi, Y., Uchida, S., Masuda, H., Kawamoto, A., Walsh, K., Isner, J. M., Asahara, T. (2001) J Clin Invest 108, 399-405
39. Vasa, M., Fichtlscherer, S., Adler, K., Aicher, A., Martin, H., Zeiher, A. M., Dimmeler, S. (2001) Circulation 103, 2885-2890
40. Rasmussen, L. M., Hansen, P. R., Nabipour, M. T., Olesen, P., Kristiansen, M. T., Ledet, T. (2001) Biochem J 360, 363-370
41. Kraynack, N. C., Corey, D. A., Elmer, H. L., Kelley, T. J. (2002) Am J Physiol Lung Cell Mol Physiol 283, L604-611
42. C. B. Treasure, J. L. Klein, W. S. Weintraub, J. D. Talley, M. E. Stillabower, A. S. Kosinski, J. Zhang, S. J. Boccuzzi, J. C. Cedarholm, R. W. Alexander, N Engl J Med 332 (1995) 481-487.
43. D. H. Walter, V. Schachinger, M. Elsner, S. Mach, W. Auch-Schwelk, A. M. Zeiher, Am J Cardiol 85 (2000) 962-968.
44. D. H. Walter, K. Rittig, F. H. Bahlmann, R. Kisrchmair, M. Silver, T. Murayama, H. Nishimura, D. W. Losordo, T. Asahara, J. M. Isner, Circulation 105 (2002) 3017-3024.
45. R. Madonna, P. Di Napoli, M. Massaro, A. Grilli, M. Felaco, A. De Caterina, D. Tang, R. De Caterina, Y. J. Geng, J Biol Chem 10 (2) (2005) 13503-13511.
46. U. Laufs, F. Custodis, M. Bohm, Drugs 66(2) (2006) 145-154.
47. M. K. Jain, P. M. Ridker, Nat Rev Drug Discov 4(12) (2005) 977-987.
48. C. P. Cavender, S. D. Turley, J. M. Dietschy, Am J Physiol 269 (1995) E331-E340.
49. D. K. Spady, J. M. Dietschy, J Lipid Res 24 (1983) 303-315.
50. M. I. Cerezo-Guisado, L. J. Garcia-Marin, M. J. Lorenzo, M. J. Bragado, J Nurochem 94 (2005) 1277-1287.
51. J. D. Barth, O. A. Kruisbrink, A. L. Van Dijk, BMJ 301 (1990) 669.
52. T. Roth, G. R. Richrdson, J. P. Sullivan, R. M. Lee, L. Merlotti, T. Roehrs, Clin Cardiol 15 (1992) 426-432.
53. O. K.-S. Lee, Y.-C. Ko, T. K. Kuo, S.-H. Chou, H.-J. Li, W.-M. Chen, T.-H. Chen, Y. Su, J Cell Biochem 93 (2004) 917-928.
54. B. W. Phillips, N. Belmonte, C. Vernochet, G. Ailhaud, C. Dani, Biochem Biophys Res Commun 284 (2001) 478-484.
55. B. W. Phillips, C. Vemochet, C. Dani, Pharmacol Res 47 (2003) 263-268.
56. X. Li, Q. Cui, C. Kao, G.-J. Wang, G. Balian, Bone 33 (2003) 652-659.
57. T. Ueyama, H. Kasahara, T. Ishiwata, Q. Nie, S. Izumo, Mol Cell Biol 23 (2003) 9222-9232.
58. T. N. Sato, Y. Qin, C. A. Kozak, L. A. Kenneth, Proc Natl Acad Sci USA 90 (1993) 9355-9358.
59. Y. M. Janumyan, C. G. Sansam, A. Chattopadhyay, N. Cheng, E. L. Soucie, L. Z. Penn, D. Andrews, C. M. Knudson, E. Yang, EMBO J 22(20) (2003) 5459-5470.
60. H. Zhou, Q. Hou, Y. Chai, Y. T. Hsu, Exp Cell Res 309(2) (2005) 316-328.
61. Kureishi Y, Luo Z, Shiojima I, et al. Nat Med. September 2000; 6(9):1004-1010
62. Rikitake Y, Liao J K. Circ Res. Dec. 9, 2005; 97(12):1232-1235.
63. Liao J K. Am J Cardiol. Sep. 5, 2005; 96(5A):24F-33F.
64. Arnaud C, Mach F. Arch Mal Coeur Vaiss. June 2005; 98(6):661-666.
65. Greenwood J, Walters C E, Pryce G, et al. Faseb J. May 2003; 17(8):905-907.
66. Kim S I, Kim H J, Han D C, et al. Kidney Int Suppl. September 2000; 77:S88-92.
67. Ledoux S, Laouari D, Essig M, et al. Circ Res. Mar. 8, 2002; 90(4):420-427.
68. Kreiselmeier N E, Kraynack N C, Corey D A, et al. Am J Physiol Lung Cell Mol Physiol. December 2003; 285(6):L1286-1295.
69. Wolf G. J Nutr Biochem. April 1999; 10(4):188-192.
70. Woodward H D, Allen J M, Lennarz W J. J Biol Chem. Feb. 15, 1988; 263(5):2513-2517.
71. Ohashi K, Osuga J, Tozawa R, et al. J Biol Chem. Oct. 31, 2003; 278(44):42936-42941.
72. Gofflot F, Hars C, Illien F, et al. Hum Mol Genet. May 15, 2003; 12(10):1187-1198.
73. Roux C, Wolf C, Mulliez N, et al. Am J Clin Nutr. May 2000; 71(5 Suppl):1270S-1279S.
74. Kelley R L, Roessler E, Hennekam R C, et al. Am J Med Genet. Dec. 30, 1996; 66(4):478-484.
75. Nowaczyk M J, Waye J S. Clin Genet. June 2001; 59(6):375-386.
76. Kelley R I, Hennekam R C. J Med Genet. May 2000; 37(5):321-335.
77. Waterham H R. FEBS Lett. Oct. 9, 2006; 580(23):5442-5449.
78. Waterham H R. Clin Genet. June 2002; 61(6):393-403.
79. Dostal L A, Schardein J L, Anderson J A. Teratology. December 1994; 50(6):387-394.
80. Surani M A, Kimber S J, Osborn J C. J Embryol Exp Morphol. June 1983; 75:205-223.
81. Carson D D, Lennarz W J. J Biol Chem. May 10, 1981; 256(9):4679-4686.
82. Carson D D, Lennarz W J. Proc Natl Acad Sci USA. November 1979; 76(11):5709-5713.
83. Liao J K. 2005 Curr Opin Lipidol. 16(6):624-629.
84. Liao J K, Laufs U. 2005 Annu Rev Phamacol Toxicol. 45:89-118.
85. Goldstein J L, Brown M S. 1986 Trans Assoc Am Physicians. 99:ccxxxi-ccxlvii.
86. Langius F A, Waterham H R, Romeijn G J, et al. Am J Med Genet A. Sep. 15, 2003; 122(1):24-29.
87. Akiba, T, et al., J Toxicol Sci. 1998; 23V:713-720.
88. Mundy G, et al. Science. 1999 Dec. 3; 286:1946-9
89. Ghosh-Choudhury N, et al. J Biol Chem, in press, 2007
90. Schultheiss T M, et al. 1997 Genes Dev, February 15; 11(4):451-62

What is claimed is:

1. A method of enhancing survival and differentiation of stem cells and cells of myogenic lineage derived from said stem cells, when said cells are exposed to an inflammatory or apoptotic stimulus, the method comprising:
culturing stem cells in vitro in a medium containing a statin, to produce statin-pretreated cells with enhanced resistance to inflammatory or apoptotic stimuli; and,
optionally, allowing said statin-pretreated cells to differentiate in the presence of said statin, to produce statin-pretreated cells of myogenic lineage.

2. The method of claim 1, further comprising exposing said pretreated stem cells, or said statin-pretreated cells of myogenic lineage, to an inflammatory or apoptotic stimulus.

3. The method of claim 1, wherein said statin is a HMG-CoA reductase inhibitor selected from the group of statin or statin-like compounds consisting of atorvastatin, cerivastatin, fluvastatin, lovastatin, mevastatin, pitavastatin, rosuvastatin, simvistatin and ezetimibe, and combinations of those.

4. The method of claim 1, wherein said statin pre-treated cells are either embryonic stem cells or adult stem cells residing in adult tissues.

5. The method of claim 1, wherein said cells of myogenic lineage comprise differentiated or undifferentiated cardiac or vascular myoblasts.

6. The method of claim 1, wherein said statin pre-treated cells are embryonic cardiac myoblasts or embryonic vascular myoblasts.

7. The method of claim 1, wherein exposing said pretreated cells to said inflammatory or apoptotic stimulus occurs in vitro, and said method further comprises determining whether survival and differentiation of said statin-pretreated stem cells is enhanced compared to non-statin-pretreated stem cells, or statin-pretreated cells of myogenic lineage derived from such stem cells, that are likewise exposed to said stimulus.

8. The method of claim 1, comprising transplanting into a diseased tissue or organ said statin pretreated stem cells, or statin-pretreated cells of myogenic lineage derived from said stem cells, in a medium comprising said statin and/or at least one isoprenoid pathway inhibitor.

9. The method of claim 1, wherein exposing said statin-pretreated cells to said inflammatory stimulus occurs in vivo, after implanting or transfusing into a host said statin-pretreated stem cells or said statin-pretreated cells of myogenic lineage derived from said stem cells.

10. The method of claim 9, further comprising administering a statin to said host prior to or after said implantation or transfusion of said pretreated cells.

11. The method of claim 1, wherein culturing stem cells in said medium containing a statin comprises culturing said cells with said statin for up to 24 hours, to produce said statin-pretreated cells.

12. The method of claim 1, wherein said medium contains said statin in a concentration of about 1 to 10 μg/mL.

13. The method of claim 1, further comprising exposing said statin-pretreated cells to at least one isoprenoid pathway inhibitor in vitro.

14. The method of claim 13, further comprising locally administering said statin- or isoprenoid pathway inhibitor-pretreated cells in vivo.

15. The method of claim 1, further comprising exposing said statin-pretreated cells to said inflammatory or apoptotic stimulus in vitro.

16. A method of protecting implanted embryonic stem cells, or cells of myogenic lineage derived from said stem cells, from the cytotoxic effects of inflammatory or apoptotic stimuli, the method comprising:
transplanting into a vascular or cardiac tissue of a mammal a plurality of in vitro statin-pretreated embryonic stem cells, or statin-pretreated cells of myogenic lineage derived from said stem cells, whereupon apoptosis in the transplanted statin-pretreated cells is inhibited.

17. The method of claim 16, wherein, prior to being transplanted or transfused, said stem cells are uncommitted and are capable of differentiating into cells having a vascular or cardiac myocyte phenotype when transplanted or transfused into said cardiac or vascular tissue.

18. The method of claim 16, wherein said stem cells are autologous or allogenic to said mammal.

19. The method of claim 16, wherein said vascular or cardiac tissue is in a mammal with heart failure or a myocardial infarction or at least one atherosclerotic lesion.

20. The method of claim 19, wherein at least one said atherosclerotic lesion is an aneurism or is an unstable plaque caused by hyperlipidemia.

21. The method of claim 16, further comprising contacting said statin-pretreated cells with a non-steroidal isoprenoid to modulate the effect of said statin.

22. The method of claim 16, wherein said tissue is exposed to at least one inflammatory agent selected from the group consisting of oxidized low density lipoprotein (oxLDL), oxysterols, cytokines and Fas ligand.

23. The method of claim 16, wherein said inhibition of apoptosis deters apoptotic cell death of said transplanted stem cells or myocytes.

24. The method of claim 16, further comprising administering clusterin to said tissue, optionally, with administration of at least one statin or at least one isoprenoid pathway inhibitor, to enhance protection of said statin-pretreated embryonic stem cells, or statin-pretreated cells of myogenic lineage derived from said stem cells, from cytotoxic effects of inflammatory or apoptotic stimuli.

25. The method of claim 16, further comprising, prior to said transplanting, treating said stem cells in a medium comprising at least one statin, at least one bone morphogenic protein and clusterin, to promote myogenic differentiation of said stem cells.
